(19) United States
(12) Patent Application Publication
(10) Pub. No.: US 2022/0008348 A1
(43) Pub. Date: Jan. 13, 2022

(54) COMPOUND DELIVERY SYSTEMS AND METHODS OF PRODUCTION

(71) Applicant: Michael Golfetto, Farmington Hills, MI (US)

(72) Inventor: Michael Golfetto, Farmington Hills, MI (US)

(21) Appl. No.: 17/292,649

(22) PCT Filed: Nov. 8, 2019
(86) PCT No.: PCT/US2019/060426

§ 371 Date: May 10, 2021

Related U.S. Application Data
(60) Provisional application No. 62/757,201, filed on Nov. 8, 2018.

(51) Int. Cl. A61K 9/51; A61K 31/352; A61K 9/00; A61K 45/06

(52) U.S. Cl. CPC A61K 9/5161; A61K 9/5176; B82Y 5/00; A61K 9/008; A61K 45/06; A61K 31/352

(57) ABSTRACT

A delivery composition including a plurality of first nanoparticle excipients each containing an active compound pharmaceutical agent and a plurality of second nanoparticle excipients each containing a modulating agent.

### CROSS-REFERENCE TO RELATED APPLICATION(S)

This U.S national phase patent application claims priority to international application no. PCT/US2019/060426, filed Nov. 8, 2019, which claims priority to and the benefit of U.S. Provisional Application Ser. No. 62/757,201, filed Nov. 8, 2018, the entire disclosures of which are hereby incorporated by reference.

### BACKGROUND

Prescription psychoactive drugs can help patients manage chronic or severe pain, restore emotional or behavioral balance, control sleep disorders, or fight obesity. When such prescription medications are abused, however, the consequences, including addiction, can be dangerous, even deadly. The risks associated with abuse of three classes of commonly abused prescription drugs, i.e., opioids; central nervous system (CNS) depressants, including sedatives and tranquilizers and OxyContin), hydrocodone (Vicodin), and meperidine (Demerol) and are commonly prescribed to relieve pain. Taken as prescribed, opioids can be used to manage pain effectively without untoward side effects. Chronic use of opioids can result in tolerance, which means that users must take higher doses to achieve the same effects. Long-term use also can lead to physical dependence and addiction. Withdrawal can occur when an individual discontinues use of the drugs. Withdrawal symptoms can include restlessness, muscle and bone pain, insomnia, diarrhea, vomiting, cold flashes with goose bumps, and involuntary leg movements. Individuals who are addicted to opioids are more likely to overdose on the drugs, which could be fatal.

### SUMMARY

This section provides a general summary of the present disclosure and is not a comprehensive disclosure of its full scope or all of its features, aspects, and objectives.

Disclosed herein are implementations of a delivery composition in accordance with aspects of the present disclosure. The delivery composition includes a plurality of first nanoparticle excipients each containing an active compound. The delivery composition further includes a plurality of second nanoparticle excipients each containing a modulating agent. Also disclosed herein are implementations of a delivery composition in accordance with aspects of the present disclosure. The delivery composition includes a plurality of nanoparticle excipients. The delivery composition further includes a plurality of microparticle excipients containing cannabidiol. The delivery composition further includes water and ethanol. Also disclosed herein is a delivery composition in accordance with aspects of the present disclosure. The delivery composition includes a dispersible concentrate configured for forming, upon contact with an aqueous solution, particles of a mean diameter of less than about 450 nm. The dispersible concentrate further includes at least one surfactant, at least one solid component at room temperature, and an amphiphilic solvent. The drug delivery composition further includes at least one Mitragyna speciosa compound selected from the group consisting of Ajmalicine, Akuammigine, Ciliaphylline, Corynantheidine, Corynoxeine, Corynoxine A, Corynoxine B, Epicatechin, 9-Hydroxycorynantheidine, 7-hydroxymitragynine, Isomitraphylline, Isomitrafoline, Isopteropodine, Isorhynchophylline, Isospeciofoline, Mitraciliatine, Mitragynine, Mitragynine oxindole B. Mitrafoline, Mitraphylline, Mitraversine, Paynantheine, Rhynchophylline, Speciociliatine, Speciofoline, Speciogynine, Speciophylline, Stipulatine, Tetrahydroalstonine, a corresponding analog, metabolite, isomer and a combination thereof, the at least one Mitragyna speciosa compound present in an amount sufficient to increase the bioavailability of the composition.

### BRIEF DESCRIPTION OF THE DRAWING

The disclosure is best understood from the following detailed description when read in conjunction with the accompanying drawings.

FIG. 1 shows an example of a microparticle excipient in accordance with aspects of the present disclosure.

### DETAILED DESCRIPTION

It is an object of the present disclosure to reduce the potential for abuse of dosage forms of psychoactive drugs and other drugs of abuse and to provide expedient systems for delivery of dosage forms of psychoactive drugs having a reduced potential for abuse.

In some embodiments of the disclosure reduction of abuse potential is achieved by proving a system having nanoparticular delivery excipient systems configured to increase bioavailability of psychotropic expedients. Increase in bioavailability of psychotropic expedients can reduce abuse potential by diminishing the concentration of psychotropic expedient or a plurality of expedients consumed necessary to achieve a minimally active dosage producing desired cognitive effect. Additionally, by increasing the bioavailability of a psychotropic expedient or plurality of expedients, synergistical alteration of an expedient pharmacokinetic and pharmacodynamic profiles has been found to reduce abuse potential by modulating both the duration of active an psychotropic expedient metabolic availability, and the desired cognitive of the expedients experienced by consumers.

Abuse of psychotropic substances is multifaceted ant thus the present disclosure discloses an excipient system with dynamic configurations intended to reduce abuse potential. Despite past innovations, consumer alteration of psychotropic dosages such as readily re-concentrating psychotropic expedients from is prescribed or standard form has led to increase abuse and addiction. Thus, some embodiments of the disclosure reduction of abuse potential is achieved by proving a system comprising of a plurality of ultrafine delivery excipient particles in a stable system that are incapable of readily being re-concentrated. Some nanoparticular delivery systems known to be capable of increasing the overall bioavailability of pharmaceutical ingredients. While increased bioavailability potentially allows for increased efficacy of psychotropic expedients desired cognitive effects, those skilled in the art will recognize that such enhanced effects can vary significantly and are dependent on the desired target drug being delivered. Additionally, increase in bioavailability can increase the potential of drugs abuse potential. Liquid dosages are especially prone to high abuse by consumers due to the lack of precision during consumption. While many attempts have been made to provide expedients with a dosage measuring system, abuse of psychotropic solutions remains a prevalent issue.

Thus, one embodiment of the present disclosure pertains to an excipient delivery system solution comprising of at least one stable psychotropic expedient in a fluidic carrier agent, where in the solution system is configured to provide minimally active dosages at concentrations which physically limit abuse potential by requiring consumers to ingest unreasonable amount of excipient solutions in order to achieve a dangerous dosage.

A non-limiting example of the present disclosure is reduced abuse liability excipient system solution containing an active non-alcoholic psychotropic expedient or pro-expedient in an amount sufficient to achieve a desired cognitive effect known to contained psychotropic expedient, wherein the maximum concentration of psychotropic expedients in the solution is about or less than 1 mg/mL.

More preferably an example of the present disclosure is reduced abuse liability nanoparticular excipient system solution containing an active non-alcoholic psychotropic expedient or pro-expedient in an amount sufficient to achieve a desired cognitive effect known to contained psychotropic expedient, wherein the maximum concentration of psychotropic expedients in the solution is about or less than 1 mg/mL.

Preferably delivery of psychotropic expedients utilizing configured nanoparticle delivery excipients has presently been found to have several distinct advantages, capable of synergistically being combined to form novel delivery systems with reduced abuse potential. Without wishing to be bound by a theory, it is believed that highly diluted delivery systems capable of reducing the required dose of psychotropic expedient necessary to achieve an effective minimally active dosage, can further simultaneously serve to proactively reduce the potential for consumers to develop tolerance to respective target psychotropic expedients.

In some embodiments the present disclosure, the disclosure further provides advantages by utilizing stable dispersions of nanoparticular excipient systems containing psychotropic expedients having poor water solubility, wherein once dispersed, nanoparticular delivery excipients in solutions are stably configured to avoid re-concentration of the excipients through means readily available to ordinary consumers. Such means otherwise available to ordinary consumers include standard filtration and centrifugation. Additionally, it has been found that stable solution systems can further be configured to prevent consumers from re-concentrating a psychotropic expedient or plurality of expedients within the systems through readily available physical, thermal and or chemical means; and attempts to do so will result in significant reduction in the overall bioavailability target psychotropic expedients through expulsion from their delivery excipients.

While some embodiments of the present disclosure relate nanoparticular delivery systems comprised of poorly water-soluble psychotropic expedients, systems disclosed herein are further capable of concomitantly delivering of water-soluble expedients as well.

In some embodiments of the present disclosure, excipient systems are configured to synergistically employ a nanoparticular excipient system comprised of at least one psychotropic expedient, a non-psychotropic agent, a pharmaceutically acceptable ingredient, or a plurality therefore of. In preferred embodiments, the expedients, agents, ingredients, or combination there for of, are herbally derived. Those skilled in the art will recognize that predictive effects of multi-drug systems delivering a plurality of psychotropic expedients are inherently complex and frequently present challenges unforeseeable complications arising from multi-drug interactions.

Unexpectedly it has been found that delivery of multiple psychotropic expedients, agents, and or ingredients utilizing modulating delivery systems comprising of at least a plurality of nanoparticular delivery excipients can increase bioavailability of individual psychotropic drug contained within the system while simultaneously reducing the dosage required to achieve a minimally active therapeutic effect of each psychotropic expedient, from traditional administration techniques.

Some aspects of the present disclosure pertain to excipient delivery systems capable of reducing abuse liability of psychotropic expedients through combined pharmacokinetic and pharmacodynamic modulation of excipients and expedient released in and around mammalian tissues.

In some embodiments of the present disclosure rapid and delayed delivery excipient systems are utilized to reduce abuse potential and modulated delivery of psychotropic expedients. In some embodiments rapid and delayed release of expedients is realized in solid oral dosages. Preferably some embodiments of the present disclosure pertain to excipient systems comprising of liquid dosages. Liquid dosage solutions may be provided in I.V. or suppository forms. More preferably liquid dosage solutions are present in an orally ingested liquid such as a beverage.

Because the rate of consumption of medicating beverages can vary drastically based off consumers preference regarding rate of drinking, modulated release of orally ingested psychotropic delivery solutions presents further challenges opposed to oral solid dosages.

Consumption of a medicating beverage having poorly water-soluble psychotropic expedients, such as expedient emulsions, can result in delayed onset time of cognitive effects. Delayed effects taking as long as 30 minutes from the time of initial consumption often lead consumers to ingest excessive amounts of beverages prior to the onset psychotropic expedients of the initial beverage. Users inadvertently ingesting an excess of a medicating solution can result in negative side effects, toxicity, and rapid development of user tolerance. Some embodiments of the present disclosure represent significant benefit; by providing an excipient system capable of rapidly releasing contents within excipients in as little as an average of about 4 minutes subsequent to initial ingestion. In some embodiments rapid and delayed delivery phases of oral excipient solutions are configured to allow consumers to gauge accurate dosing and provide or extend desired cognitive effects of psychotropic expedients as needed. In some embodiments of the present disclosure, excipient systems may be formulated to have accelerated clearance rates, allowing for rapid dissipation of positive or negative cognitive effects in as little as 30 minutes subsequent to ingestions.

In some embodiments of the present disclosure, a modulating excipient system comprising a nanovesicle encompassing at least one psychotropic active ingredient, is utilized as a means of limiting abuse potential of psychoactive excipients by regulating expedients delivery.

In some embodiments, the present disclosure utilizes a plurality of stable nanoparticle excipients having an average diameter of less than or about 450 nm, and a plurality stable microparticle excipients having an average diameter of no greater than about 1500 nm to regulate the delivery of psychotropic excipients over periodic consumption of a single dosage or multiple dosages.

In some embodiments, regulation of psychotropic excipients is achieved through excipients configured to temporally control release of psychotropic expedients. In some embodiments of the present disclosure, excipient systems are comprised of a quick release excipient, a delayed release excipient, and a latent expedient or agent. In some embodiments of the disclosure, latent expedients or agents are utilized to provide a physiological, psychological, pharmacokinetic, or pharmacodynamic means of reducing abuse liability through reduction in association f drug seeking behavior and stress inducing ques sufficient to induce the behavior.

In some embodiments, excipient delivery systems comprising of at least a plurality of stable nanoparticles and at least one psychotropic expedient is utilized in conjunction with a Pgp-efflux modulator, an enzyme modulator, a receptor modulator, or a combination there for of.

In some embodiments Pgp-efflux modulator may be comprised of a competitive or non-competitive substrate inhibitor or inducer of a psychotropic expedient present in the same dosage.

In some embodiments enzymatic modulator may be comprised of a competitive or non-competitive substrate inhibitor or inducer of a psychotropic expedient present in the same dosage.

In some embodiment's receptor modulator may in relation to the psychotropic expedient present in the same dosage, be comprised of a competitive, non-competitive, allosteric inhibitor or inducer, or a plurality therefore comprising of a partial agonist, a agonist, a partial agonist, an antagonist or a combination therefore of.

In some embodiments of the present disclosure, an abuse limiting, or reduced abuse liability excipient system is comprised of no active psychotropic drugs is utilized to deliver a safe dose of excipients or agents in an amount sufficient to reduce the abuse potential of another psychotropic or addictive expedient not contained within an excipient system dose.

In some embodiments of the present disclosure, a dose of an abuse limiting, or reduced abuse liability excipient system is comprised of an amount of a psychotropic expedient insufficient to induce full cognitive effects commonly associated with the psychotropic expedient, but in amounts sufficient to enhance the bioavailability of another easily abused quantity of a psychotropic expedient not present in excipient system dose, or reduce the need for consumption therefore of.

In some embodiments of the present disclosure, a modulator may be comprised of an additional psychotropic expedient different from a first molecularly psychotropic expedient, a non-psychotropic expedient, an active or inactive agent, a pharmaceutically acceptable ingredient, or a combination thereof.

While expedients referenced herein primarily refer to psychotropic active substances primarily targeting g coupled protein receptors, the term “expedients” is used may further extend to other classes of psychotropic drugs as well as non-psychotropic pharmaceutical ingredients primarily targeting non-g-coupled protein receptors.

As used herein, the term “Abuse limiting excipient system” refers to an excipient system comprising of a total psychotropic expedient content of less than 10 mg per/mL of system.

As used herein, the term “reduced abuse limiting excipient system” refers to a excipient system comprising of a total psychotropic expedient content of less than 10 mg per/mL of system and may be used interchangeably with “reduced abuse excipient system” or “reduce abuse liability system” without limitation.

As used herein, the term “psychotropic” and “psychoactive” expedient may be used interchangeably and refers to chemical substance that changes brain function and results in alterations in perception, mood, consciousness, cognition, or behavior and comprised of synthetically or naturally derived expedients preferably including but not limited to: 2-aminoethanesulfonic acid, 2-Fluorohmefentanil, 3-Methylfentanyl, 4-Phenylfentanyl, 4-carboethoxyohmefentanil, 14-Cinnamoyloxycodeinone, Acetorphine, Acetylfentanyl, Acetylpropionylmorphine, Actinidine, Aporphine, Ajmalicine, Akuammidine, yr-Akuammigine, Alfentanil, Alfentanyl, Alfentanil, Amphetamine, Arecoline, Asarone, Ayahuasca extracts, Baicalein, Beta-Casomorphine-7, Benzodiazepine, BDPC, Benzoylmethylecgonine, Brifentanil, Buprenorphine, Butyrfentanyl, Cannabidiol, Cannabidiolic Acid, Cannabinol, Cannabigerol, Cannabichromene, Cannabicyclol, Cannabivarin, Cannabidivarin, Cannabichromevarin, Cannabigerovarin, Cannabigerol Monomethyl Ether, Cannabielsoin, Cannabicitran, Carfentanil, Carnitine, Clonitazene, Codeine, Corynantheidine, Corynoxeine, Corynoxine A, Corynoxine B, Chrysin, Delosperma harazianum, a Dehydromethysticin, Desmethoxyyangoni, Desomorphine, Dezocine, Dextroamphetamine, Diamorphine, Diacetylmorphine, Diacetyldihydromorphine, Dibenzoylmorphine, Dihydrocodein, Dihydroetorphine, Dihydrodesoxy morphine, a Dihydromethysticin, a Dihydrokavain, a Dihydroyangonin, Dipropionate, a Diterpene, Enadoline, an Epicatechin, Ethanol, Etonitazene, Etorphine, Fentanyl, Flavokavain A, Flavokavain B, Flavokavain C, Furanylfentanyl, a Guarana constituent, a Ginsenoside′ a Hemiterpene, a Hydroxyavain, Hydrocodone, Hydromorphone, a Hydroxycorynantheidine, a Hydroxydehydrokavain, a Hydroxymitragynine, a Hydroxyyangonin, Hyperforin, an Iboga alkaloid, Isomitraphylline, Isopteropodine, Kavain, a Kavalactone, Lactucin, Lactucopicrin, Lagochilin, Leonurine, Levoamphetamine, Levorphanol, Lofentanil, Lobeline, Lysergic acid, Lysergic acid diethylamide, Mesembrine, Methadone, a Methoxyyangonin, Methoxy-12-hydroxydehydrokavain, Methyldesorphine, Methysticin, Mitragynine, Mitraphylline, Mitajavine, a Monoacetylmorphine, a Monoterpene, Morphine, Morphine dinicotinate, Myrcene, Myristicin, Nicomorphine, Nicotine, N-methylphenethylamine, a Norisoprenoid, N-Phenethyl-14-ethoxymetopon, N-Phenethylnordesomorphine, N-Phenethylnormorphine, Ocfentanil, Ohmefentanyl, Oxycodone, Oxymorphol, Oxymorphone, Papaverine, Paynantheine, Pethidine, Phenaridine, Phenazocine, Phenethylamine, Phenomorphan Phenibut, a Polyterpene, Pukateine, Psilocybin, Remifentanil, a Rhodiola Rosea extract, Rhynchophylline, a Sesterterpene, a Sesquiterpene, a Sesquarterpene, Speciociliatine, Speciogynine, Speciophylline, Sufentanil, a Tetraterpene, a Tetrahydroyangonin, a Tetrahydroalstonine, a Tetrahydrocannabinol, a Tetrahydrocannabinolic acid, a Tetrahydrocannabivarin, Thebaine, Trefentanil, a Triterpene, Voacangine, Yangonin; Yohimbine; an metabolite of any of the aforementioned expedients an isomer of any of the aforementioned expedients; an analogue any of the aforementioned expedients; a derivative of any of the aforementioned expedients; a salt of any of the aforementioned expedient; or a combination therefore of.

The compounds used in the method of the present disclosure may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkali metal salts, sodium, potassium or lithium.

The term “nanoparticular excipient” and or “nanoparticle excipient” may be used in the present disclosure interchangeably, and here in refer to a particle having a mean diameter of about 450 nanometers or less.

The term “nanoparticular excipient system” and or “nanoparticle excipient system” may be used in the present disclosure interchangeably, and here in refer to a particle having a mean diameter of about 800 nanometers or less.

As reference here in “stable nanoparticle” a carrier matrix having structural integrity sufficient to maintain an individual nanoparticles dimensions in a range consistent with the averaged size of similar nanoparticle excipients concomitantly populating an excipients system dose, at temperature of at least 45 degrees Celsius and below.

“Average size” is understood as the average diameter of the population of excipient nanoparticles. The average size can be measured by standard methods known by the person skilled in the art and described, for example, in the example section below.

The terms “cell membranes” and “biological barriers” in this disclosure refer to 1) the mucosal membrane barriers of the oral cavity; 2) the mucosal membrane barrier of the GI tract; 3) the dermal and epidermal cell membrane barriers; 4) the BBB; 5) the blood-ocular barrier consisting of the blood-aqueous barrier and the blood-retinal barrier; 6) ocular barriers of the conjunctiva and corneal epithelium; and 7) the cell membrane barriers of the nervous system, respiratory system, circulatory system, GI system, muscular system, urinary system, genital system, internal organs, and tissues.

The phrase “pharmaceutically acceptable carrier” as used herein means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject water soluble inclusion complex from one organ, or portion of the body, to another organ, or portion of the body. Each carrier must be “acceptable” in the sense of being compatible with the other ingredients of the formulation and not injurious to the patient. Pharmaceutically acceptable carriers may include, for example, an adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. For tableting, fillers and excipients such as lactose and calcium bicarbonate may be added.

The term “polymer” refers to molecules formed from the chemical union of two or more repeating units, called monomers. Accordingly, included within the term “polymer” may be, for example, dimers, trimers and oligomers. The polymer may be synthetic, naturally-occurring or semisynthetic. In preferred form, the term “polymer” refers to molecules which typically have a Mw greater than about 3000 and preferably greater than about 10,000 and a Mw that is less than about 10 million, preferably less than about a million and more preferably less than about 200,000. Examples of polymers include but are not limited to, poly-a-hydroxy acid esters such as, polylactic acid (PLLA or DLPLA), polyglycolic acid, polylactic-co-glycolic acid (PLGA), polylactic acid-co-caprolactone; poly (ester-co-amide) copolymers; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

The “dispersible concentrate” is a composition which spontaneously forms a nano-particulate dispersion in an aqueous medium, for example in water upon dilution, or in the gastric juices after oral administration. The “dispersible concentrate” includes those compositions that form solid particles having a mean diameter of less than about 500 nm upon contact with an aqueous medium.

The “aqueous medium,” refers to a water based medium, i.e., a liquid medium in which water is the major component. In accordance with the present disclosure, the aqueous medium may be the digestive fluid formed in the stomach (e.g., gastric fluid formed by cells lining the stomach), GI tract fluids or any liquid medium, in vivo or ex vivo in which the herein defined dispersible concentrate is dissolved.

The terms “formulation” and “composition” may be used interchangeably. A “unit dose formulation” or “unit dose form” is a formulation or composition in a single dose size comprised of excipient systems disclosed herein. Non-limiting examples include pills, tablets, caplets, capsules, slurries, liquids, suspensions, etc.

The term “modulating agent” means any material employed which has biological, chemical, physiological, pharmacokinetic or pharmacodynamic utility comprising of at least one or more functions including, without limitation, altering, diminishing, enhancing, potentiating, inducing, inhibiting, regulating, maintaining, prolonging, or reducing the activity of an expedient, drug, active agent, pharmaceutical ingredient, active substance, pharmaceutically acceptable carrier, tissue structures, receptors, enzymes, substrates, biological membranes, or ligands, or a plurality therefore of.

The term “ingredient” or “agent” may be used interchangeably, and herein refers to any compound or substance which has biological, chemical, or physiological efficacy including, without limitation, an active pharmaceutical ingredient, drug, naturally occurring compound, nucleic acid compound, peptide compound, biologic, nutraceutical, agricultural or nutritional ingredient, or expedient.

As used herein, the term “expedient” or “expedients” refers to, without limitation, a synthetic drug, naturally occurring drug, psychotropic expedient, non-psychotropic expedient, or herbal extract, or a combination thereof; including but not limited too addictive substances such as opioid agonists or narcotic analgesics, hypnotics, tranquilizers, stimulants and antidepressants.

The terms “primary” and “secondary” used in conjunction with “ingredient” were used to assist simply for antecedent purposes and are not meant to imply the level of importance of the active ingredient.

If the term “surrounding” is used alone, without any qualifier, it is understood to mean “at least partially surrounding”.

The terms “encapsulating” and “encompassing” may be used interchangeably in this application and are defined for purposes of the present disclosure without limitation as container or carrier.

The term “encapsulate” in this disclosure refers to coating of various substances within another material. The encapsulated material is referred to as the internal phase, the core, or the fill material. The encapsulation material is known as the external phase, the shell, coating or membrane.

The term “payload” in this disclosure refers materials such as but not limited to, excipients, modulating agents, drugs, pharmaceutical ingredients, or fillers delivered by a portion of one or more excipient systems.

The term “ligand” in this disclosure refers to any material that may be bound to the surface of the nanoparticle or nanostructure for the linking of nanoparticles to form nanometer-scale geometric structures.

The term “viscoelastic” in this disclosure refers to the simultaneous existence of viscous and elastic properties of nanoparticles and their behavior thereof from intermolecular and interparticle forces in their compositional material.

The term “biocompatible” in this disclosure refers to the ability of nanoparticle compositions and biomaterials to perform their desired functions without eliciting any undesirable local or systemic effects in the recipient, generating the most appropriate beneficial cellular and tissue responses and optimizing the performance of their payloads. This is especially relevant on the nanoscale where biomaterials function differently can introduce undesirable, adverse and sometimes toxic effects.

The term “biodegradable” in this disclosure refers to the ability of nanoparticle compositions and biomaterials to rapidly metabolize in vivo and resulting metabolites that are nontoxic and readily eliminated.

The term “surfactant” in this disclosure refers to compounds that lower the surface tension (or interfacial tension) between two liquids or between a liquid and a solid act as emulsifiers, dispersants, wetting agents and viscosity modifiers. In one embodiment surfactants means amphiphilic molecules which are manufactured by chemical processes or purified from natural sources or processes that can be anionic, cationic, nonionic, and zwitterionic.

The term “controlled release” may be variously characterized by “sustained release”, “sustained action”, “extended release”, “modified release”, “pulsed release”, “quick release”, “delayed release”, “targeted release”, “site specific release”, and “timed release”, which are used interchangeably in this application and are defined for purposes of the present disclosure as the time of release, the extent of release, the rate of release, the site of release and/or release of an active ingredient from a formulation at such a rate that when a dose of the active ingredient is administered in the sustained release, extended release, pulsed release, timed release, quick, delayed release or controlled-release.

As used herein, the term “deliver” or “delivery” may be used interchangeably in this application and are defined for purposes of the present disclosure without limitation as disposing, depositing, releasing, or otherwise making available to available to mammalian tissues.

The term “inhibit” refers to partially, substantially, or completely slowing, hindering, reducing, delaying or preventing. The terms inhibit, reduced, prevented, delayed, and slowed may be used interchangeably.

The term “environmental stimuli” here in refers to, without limitation, stimuli located in an immediate vicinity comprised of thermodynamic, chemical, radiational, physiological, biological, electromagnetic, or other known in the art, or a combination thereof.

In accordance with the present disclosure, the administration of the therapeutic agent pertaining to the other therapeutic modality can be carried out by any appropriate route including, but not limited to, oral routes, intravenous routes, intramuscular routes or any other administration route that is suitable to achieve the desirable effect of the other therapeutic modality.

Formulations suitable for oral administration may comprise (a) liquid solutions, such as an effective amount of the composition dissolved in a nonaqueous diluent; (b) capsules, sachets, tablets, lozenges, and troches, each containing a predetermined amount of the composition, as solids or granules; (c) powders; (d) suspensions in an appropriate non-aqueous liquid; and (e) suitable non-aqueous emulsions. Liquid formulations may include diluents, such as alcohols, for example, ethanol, benzyl alcohol, and the polyethylene alcohols, either with or without the addition of a pharmaceutically acceptable surfactant, suspending agent, or emulsifying agent. Capsule forms may be of the ordinary hard- or soft-shelled gelatin type containing, for example, surfactants, lubricants, and inert fillers, such as lactose, sucrose, calcium phosphate, and corn starch. Tablet forms may include one or more of lactose, sucrose, mannitol, corn starch, potato starch, alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms may comprise the composition in a flavor, usually sucrose or acacia as well as pastilles comprising the composition in an inert base, such as gelatin and glycerin, or sucrose and acacia, emulsions, gels, and the like containing.

Abuse Limiting Excipient System

In some embodiments of the present disclosure, an abuse limiting excipient system is a dose comprised of at least a plurality of first nanoparticle excipients encompassing a first expedient and a second excipient containing an expedient or agent in an amount sufficient to increase the bioavailability of the first expedient, where in the concentration of the first expedient in the excipient system is less than 100 mg per mL, or volumetric equivalent therefore of.

In some embodiments of the present disclosure, an abuse limiting excipient system is a dose comprised of at least a plurality of first nanoparticle excipient encompassing a first alcoholic psychotropic expedient and a second excipients containing an expedient or agent in an amount sufficient to increase the bioavailability of the first psychotropic expedient, where in the concentration of the first psychotropic expedient in the excipient system is less than 100 mg per mL, or volumetric equivalent therefore of.

Preferably some embodiments of the present disclosure, an abuse limiting excipient system is a dose comprised of at least a plurality of first nanoparticles excipient encompassing a nonalcoholic first psychotropic expedient and a second excipient containing an active agent in an amount sufficient to increase the bioavailability of the first psychotropic expedient, where in the concentration of the first psychotropic expedient in the excipient system is less than 10 mg per mL, or volumetric equivalent therefore of.

In preferred embodiments, an abuse limiting excipient system is a dose comprised of a plurality of nanoparticle excipients delivering expedients, agents, or a combination therefore of wherein the concentration of an expedient or agent contained within the abuse limiting excipient system is less than 10 mg/mL; and wherein expedients or agents are present in an amount sufficient to reduce the need for excessive consumption of a psychotropic expedient or the resulting deleterious effects therefore of

Reduced Abuse Liability Excipient Systems

In some embodiments of the present disclosure, a reduced abuse liability excipient system dose is comprised of at least a plurality of first nanoparticle excipient encompassing a first expedient and at least a second excipient containing an expedient or agent in an amount sufficient to increase the bioavailability of the first expedient, where in the concentration of the first psychotropic expedient in the excipient system is less than 1 mg per mL, or volumetric equivalent therefore of.

In preferred embodiments, an reduced abuse liability excipient system dose is comprised of at least plurality of nanoparticle excipients delivering expedients, agents, or a combination therefore of wherein the concentration of an expedient or agent contained within the reduced abuse liability excipient system is less than 1 mg per mL; and wherein expedients or agents are present in in an amount sufficient to reduce the need for excessive consumption of a psychotropic expedient or the resulting deleterious effects therefore of.

Some embodiments of the present disclosure pertain to a dose of an abuse limiting or reduced abuse liability excipient system comprised of at least a plurality of excipient nanoparticles having a mean diameter of about 450 nm or smaller; wherein a dose of the excipient systems contains a population of nanoparticle excipients delivering one or more expedients in an amount equal to or greater than about 0.0001 μmoles and less than or equal to about 0.769 moles per a dose of the excipient systems; wherein a delivered expedient is comprising a corresponding amount of a compound comprised of: 2-aminoethanesulfonic acid, 2-Fluorohmefentanil, 3-Methylfentanyl, 4-Phenylfentanyl, 4-carboethoxyohmefentanil, 14-Cinnamoyloxycodeinone, Acetorphine, Acetylfentanyl, Acetylpropionylmorphine, Actinidine, Aporphine, Ajmalicine, Akuammidine, ψ-Akuammigine, Alfentanil, Alfentanyl, Alfentanil, Amphetamine, Arecoline, Asarone, Ayahuasca extracts, Baicalein, Beta-Casomorphine-7, Benzodiazepine, BDPC, Benzoylmethylecgonine, Brifentanil, Buprenorphine, Butyrfentanyl, Cannabidiol, Cannabidiolic Acid, Cannabinol, Cannabigerol, Cannabichromene, Cannabicyclol, Cannabivarin, Cannabidivarin, Cannabichromevarin, Cannabigerovarin, Cannabigerol Monomethyl Ether, Cannabielsoin, Cannabicitran, Carfentanil, Carnitine, Clonitazene, Codeine, Corynantheidine, Corynoxeine, Corynoxine A, Corynoxine B, Chrysin, Delosperma harazianum, a Dehydromethysticin, Desmethoxyyangoni, Desomorphine, Dezocine, Dextroamphetamine, Diamorphine, Diacetylmorphine, Diacetyldihydromorphine, Dibenzoylmorphine, Dihydrocodein, Dihydroetorphine, Dihydrodesoxy morphine, a Dihydromethysticin, a Dihydrokavain, a Dihydroyangonin, Dipropionate, a Diterpene, Enadoline, an Epicatechin, Ethanol, Etonitazene, Etorphine, Fentanyl, Flavokavain A, Flavokavain B, Flavokavain C, Furanylfentanyl, a Guarana constituent, a Ginsenoside, a Hemiterpene, a Hydroxyavain, Hydrocodone, Hydromorphone, a Hydroxycorynantheidine, a Hydroxydehydrokavain, a Hydroxymitragynine, a Hydroxyyangonin, Hyperforin, an Iboga alkaloid, Isomitraphylline, Isopteropodine, Kavain, a Kavalactone, Lactucin, Lactucopicrin, Lagochilin, Leonurine, Levoamphetamine, Levorphanol, Lofentanil, Lobeline, Lysergic acid, Lysergic acid diethylamide, Mesembrine, Methadone, Methoxetamine, a Methoxyyangonin, Methoxy-12-hydroxydehydrokavain, Methyldesorphine, Methysticin, Mitragynine, Mitraphylline, Mitajavine, a Monoacetylmorphine, a Monoterpene, Morphine, Morphine dinicotinate, Myrcene, Myristicin, Nicomorphine, Nicotine, N-methylphenethylamine, a Norisoprenoid, N-Phenethyl-14-ethoxymetopon, N-Phenethylnordesomorphine, N-Phenethylnormorphine, Ocfentanil, Ohmefentanyl, Oxycodone, Oxymorphol, Oxymorphone, Papaverine, Paynantheine, Pethidine, Phenaridine, Phenazocine, Phenethylamine, Phenomorphan Phenibut, a Polyterpene, Pukateine, Psilocybin, Remifentanil, a Rhodiola Rosea extract, Rhynchophylline, a Sesterterpene, a Sesquiterpene, a Sesquarterpene, Speciociliatine, Speciogynine, Speciophylline, Sufentanil, a Tetraterpene, a Tetrahydroyangonin, a Tetrahydroalstonine, a Tetrahydrocannabinol, a Tetrahydrocannabinolic acid, a Tetrahydrocannabivarin, Thebaine, Trefentanil, a Triterpene, Voacangine, Yangonin; Yohimbine; an isomer of any of the the expedients; an analogue any of the the expedients; a derivative of any of the expedients; a salt of any of the expedients, a metabolite of the expedients; or a combination therefore of.

Modulated Excipient Systems

In one embodiment of the present disclosure, an abuse limiting or reduced abuse liability excipient system is comprised of an excipient system readily being concomitantly consumed comprising of at least a of a plurality of first nanoparticle excipient encompassing a first psychotropic expedient and a plurality of second excipients containing an modulating agent; wherein the modulating agent is comprised of an inhibitor or inducer in an amount sufficient to increase the bioavailability of the first psychotropic expedient.

In some embodiments of the present disclosure, a modulating agent is a P-glycoprotein inhibitor. In some embodiments of the present disclosure, a modulating agent is a comprised of at least one competitive, mixed, noncompetitive CYP450 inhibitor, or a combination therefore of.

In some embodiments of the present disclosure, the modulating agent is a CYP-2C9 inhibitor. In some embodiments of the present disclosure, the modulating agent is a CYP-2D6 inhibitor. In some embodiments of the present disclosure, the modulating agent is a CYP-3A4 inhibitor. In some embodiments of the present disclosure, the modulating agent is a UG72B7 inhibitor.

Preferably some embodiments pertain to an abuse limiting or reduced abuse liability excipient system utilizing a concomitantly administered modulating agent; wherein the modulating agent is comprised of an inhibitor or inducer in an amount sufficient to increase the bioavailability of the first psychotropic expedient while limiting the undesired deleterious effects of the psychotropic expedient, agents, a plurality therefore of, or a combination thereof.

In other embodiments of the present disclosure, the modulating agent is a P-glycoprotein inhibitor with an EC 50 of less than about 34.5±4.2 μM.

In some embodiments of the present disclosure, a modulating agent is a comprised of at least one competitive, mixed, noncompetitive CYP450 inhibitor, or a combination therefore of.

Preferably in some embodiments, modulating agent is a comprised of at least one competitive, mixed, noncompetitive CYP450 inhibitor, or a combination therefore of; having an IC50 value about or less than 43.2±6.2 μM.

In some specific embodiments of the present disclosure, the modulating agent is comprised of a CYP-2C9 inhibitor having an IC50 value about or less than 32. 1±3.7 μM, a CYP-2D6 inhibitor having an IC50 value about or less than 27. 4±5.3 μM, a CYP-3A4 inhibitor having an IC50 value about or less than 43.2±6.2 μM, or a combination thereof.

In some embodiments, an abuse limiting or reduced abuse liability excipient system is comprised of an excipient system readily being concomitantly consumed comprising of at least a of a plurality of first nanoparticle excipient encompassing a first psychotropic expedient and a plurality of second excipients containing a plurality of modulating agents; where in the modulating agent is comprised of a P-glycoprotein inhibitor, a CYP-450 inhibitor having an IC-50 value less than 49.8 μM, a UG72B7 inhibitor, or a combination thereof acting as competitive, mixed, or non-competitive inhibitors, or a combination thereof in an amount sufficient to increase the bioavailability of the first psychotropic expedient.

In some embodiments of the present disclosure, an abuse limiting or reduced abuse liability excipient system is comprised of an excipient system comprising of at least a plurality nanoparticle excipients delivering a payload comprised of a fist expedient and at least an excipient delivering a modulating agent comprising of a 5-HT2A, 5-HT3A, NMDA, ADORA1, ADORA2A, ADORA2B, or a ADORA3 receptor antagonist; or a plurality therefore of.

In some embodiments of the present disclosure, an abuse limiting or reduced abuse liability excipient system is comprised of an excipient system comprising of at least a plurality nanoparticle excipients delivering a payload comprised of a fist expedient and at least an excipient delivering a modulating agent comprising of a selective μ-opioid agonist (for increasing the bioavailability of the opioid while reducing the quantity of opioid required), partial μ-opioid agonist, antagonist

Time Modulated Excipient System

In some embodiments of the present disclosure, an abuse limiting or reduced abuse liability excipient system is comprised of part of a dose at least a quick delivery excipient system comprising of a plurality of first nanoparticle excipients encompassing a first psychotropic expedient; and a plurality of delayed delivery excipients containing at least one modulating agent in an amount sufficient to alter the bioavailability of the first psychotropic expedient for about at least 5 or more minutes; wherein the quick delivery excipient system is capable delivering its contents at an enhanced rate of about 30 seconds or more than concurrently administered delayed delivery excipient. Quick and delayed delivery excipients may have a delivery time differential arising from excipient particle size, surface functionalization, structural functionality, reactivity towards proximal environmental stimuli, delivery route, or a combination therefore of.

In some embodiments of the present disclosure, a delivery excipient system is configured to preemptively deliver a preponderance of a first psychotropic expedient 30 seconds-12 hours prior to the delivery of a preponderance of the delayed delivery excipients payloads. In some embodiments, the excipient systems delayed delivery excipients payloads are comprised of an additional amount of a psychotropic expedient, a modulating agent, a pharmaceutical ingredient, or a combination there for of Modulating agent payloads may be further comprised of at least a competitive, a mixed, a noncompetitive inducing or inhibiting agent, or a combination there for of, effecting a P-glycoproteins pump, a CYP-450 substrate, a UG72B7 substrate, a drug receptor, a neurotransmitter, or a combination thereof.

In some embodiments of the present disclosure, an abuse limiting or reduced abuse liability excipient system is comprised of at least a quick delivery excipient system comprising of a plurality of first nanoparticle excipients encompassing modulating agent; and a delayed excipient system comprising at least a plurality of delayed delivery excipients containing a first expedient; where in the quick delivery excipient system is capable delivering preponderance of its contents in an amount sufficient to alter the bioavailability, at an enhanced rate of about 30 seconds or more than concurrently administered delayed delivery excipients. Quick and delayed delivery excipients may have a delivery time differential arising from excipient particle size, surface functionalization, structural functionality, reactivity towards proximal environmental stimuli, delivery route, or a combination therefore of.

In some embodiments of the present disclosure, a delivery excipient system is configured to preemptively deliver a preponderance of a modulating agents 30 seconds 12 hours prior to the delivery of a preponderance of the systems delayed delivery excipient payload. In some embodiments, the excipient systems delayed delivery excipients payloads are comprised of an additional amount of a psychotropic expedient, a modulating agent, a pharmaceutical ingredient, or a combination there for of Modulating agent payloads may be further comprised of at least a competitive, a mixed, a noncompetitive inducing or inhibiting agent, or a combination there for of, effecting a P-glycoproteins pump, a CYP-450 substrate, a UG72B7 substrate, a drug receptor, a neurotransmitter, or a combination thereof.

In some embodiments of the present disclosure, a delivery excipients contents may be comprised of a pharmacodynamic modulating agent reversibly or irreversibly effecting an Adrenergic, Dopaminergic, GABAergic, Glutaminergic, Cholinergic, Muscarinic, Nicotinic, Opioid, Serotonergic, Glycinergic, or a Cannabinoid receptor, or a plurality therefore of. Furthermore, a modulating agent's mechanisms may include but not be limited to allosteric, selective, non-selective, inverse agonistic, partial agonistic, agonistic, or partial antagonistic modes of action, or a plurality therefore of.

In some preferable embodiments of the present disclosure, an abuse limiting or reduced abuse liability excipient system is comprised of an excipient system comprising of at least a plurality nanoparticle excipients delivering a payload comprised of a fist quick delivered expedient and at least an delayed delivery excipient delivering a modulating agent comprising of a antagonist effecting a 5-HT2A, 5-HT3A, NMDA, ADORA1, ADORA2A, ADORA2B, or ADORA3 receptor; or a combination thereof.

In some preferable embodiments of the present disclosure, an abuse limiting, or reduced abuse liability excipient system is comprised of an excipient system comprising of at least a plurality nanoparticle excipient delivering a payload comprised of a fist expedient and at least an excipient delivering a modulating agent comprising of a selective μ-opioid agonist, partial μ-opioid agonist, opioid antagonist; or a plurality therefore of.

In other embodiments of the present disclosure, an abuse limiting, or reduced abuse liability excipient system is comprised of at least a quick delivery excipient system comprising of a plurality of first nanoparticle excipients encompassing a first expedient or first modulating agent; and a delayed excipient system comprising at least a plurality of delayed delivery excipients, and a latent delivery expedient system. Latent delivery expedient systems are comprised of a plurality of nanoparticular excipients utilized for the delivery an excipient or agent having a portion the expedients or agents stored in and around mammalian tissues for prolonged duration of time, wherein the excipients or agents are present in the amount sufficient to prolongedly reduce drug seeking behavior associated with a psychotropic or addictive substance. In some embodiments of the disclosure, a portion of a latent expedient or agent is distributed in and around lipophilic mammalian tissues for a duration of at least 4 hours and lasting as long as 240 hours or more. In some embodiments of the disclosure, a portion of latent expedients or agents stored prolongedly distributed for 4 or more hours is about or more than 5 wt % of the total amount present in a corresponding excipient system dose at the time of consumption.

Excipient Systems Structure

In some embodiments, psychotropic expedients and or agents are contained within a liposome, a micellular shell, a solid lipid matrix, a lipid monolayer, or lipid bilayer matrix, a glycol layer matrix, an inorganic layer matrix, an inorganic polymer layer matrix, and organic polymer layer matrix, a protein matrix, a polysaccharide matrix, a plastic matrix, a semisolid matrix, a gel matrix, a sol-gel matrix, a wax matrix, complex matrix, or a combination there fore of, preferably having diameter of about 450 nm or smaller. In some embodiment of the present disclosure, a nanoparticle excipient is comprised of about 0.21-1200 zeptograms of at least on expedient.

In some embodiments, excipient nanoparticles are a delivered in self nanoemulsifying drug delivery systems (SNEEDS). SNEDDS maybe be comprised of solid, liquid, or gel concentrates. SNEDDS concentrates may be encapsulated in a carrier such as but not limited to, a gel capsule or a porous carrier matrix, or be provided in a readily consumable liquid concentrate. Other non-limiting formations may be administered in a transdermal system such as a gel, lubricant, lotion, spray, or transdermal patch. Additional formulations may include, without limitation, gums, elixirs, candy, soft gels, capsules, eyedrops, nasal sprays, dry nasal powders, inhalant formulations, intravenously administered formulations, or other known oral and parental administration method formulations.

In some embodiments, psychotropic expedients and or agents are contained within the macro, meso, or microporous structure of delivery nanoparticles. In the embodiments, porous nanoparticles are comprised of organic polymers, or inorganic polymers or a combination thereof. In some embodiments, the porous matrix may be an aerogel. The porous matrix is composed of any organic or inorganic material known in the art, such as, silica, metal and metalloid oxides, metal chalcogenides, metals, metalloids, amorphous carbon, graphitic carbon, diamond, discrete nanoscale objects, organic polymers, biopolymers, polyurea, a polyurethane, a polyisocyanate, a polyisocyanurate, a polyimide, a polyamide, a polybenzoxazine, a polyacrylonitrile, a polyetheretherketone, a polyetherketoneketone, a polybenzoxazole, a phenolic polymer, a resorcinol-formaldehyde polymer, a melamine-formaldehyde polymer, a resorcinol-melamine-formaldehyde polymer, a furfural-formaldehyde polymer, an acetic-acid-based polymer, a polymer-crosslinked oxide, a silica-polysaccharide polymer, a silica-pectin polymer, a polysaccharide, amorphous carbon, graphitic carbon, graphene, diamond, boron nitride, an alginate, a chitin, a chitosan, a pectin, a gelatin, a gelan, a gum, a cellulose, a virus, a capsid, a biopolymer, an ormosil, an organic-inorganic hybrid material, a rubber, a polybutadiene, a poly(methyl pentene), a polypentene, a polybutene, a polyethylene, a polypropylene, a carbon nanotube, a boron nitride nanotube, graphene, two-dimensional boron nitride, and combinations thereof as non-limiting examples. In some embodiments, suitable matrix materials may be reinforced with a fiber, a fibrous batting, aligned fibers, chopped fibers, or another suitable material. In some of these embodiments, the fiber comprises silica, glass, carbon, a polymer, poly(acrylonitrile), oxidized poly(acrylonitrile), poly(p-phenylene-2,6-benzobisoxazole) (e.g., ZYLON® polyoxazole manufactured by Toyobo Corp. (Japan)), poly(paraphenylene terephthalamide) (e.g., KEVLAR® para-aramid manufactured by DuPont (Wilmington, Del.)), ultrahigh molecular weight polyethylene (e.g., SPECTRA® ultrahigh molecular weight polyethylene manufactured by Honeywell (Morris Plains, N.J.) or DYNEEMA® ultrahigh molecular weight polyethylene manufactured by Royal DSM (Netherlands)), poly(hydroquinone diimidazopyridine) (e.g., M5), polyamide (e.g., NYLON®), natural cellulose, synthetic cellulose, silk, viscose (e.g., rayon), a biologically-derived fiber, a biologically-inspired fiber, a ceramic, alumina, silica, zirconia, yttria-stabilized zirconia, hafnia, boron, metal/metalloid carbide (e.g., silicon carbide), metal/metalloid nitride (e.g., boron nitride), nanotubes, carbon nanotubes, carbon nanofibers, boron nitride nanotubes, oxide nanotubes as non-limiting examples. Metalloids include boron, silicon, germanium, arsenic, antimony, tellurium, polonium and combinations thereof as non-limiting examples. Metals include lithium, sodium, potassium, rubidium, cesium, francium, beryllium, magnesium, calcium, aluminum, scandium, titanium, vanadium, chromium, manganese, iron, cobalt, nickel, copper, zinc, gallium, yttrium, zirconium, niobium, molybdenum palladium, silver, cadmium, indium, tin, lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold the transactinide metals and combinations thereof as non-limiting examples. Discrete nanoscale objects include carbon nanotubes, boron nitride nanotubes, viruses, semiconducting quantum dots, graphene, and combinations thereof as non-limiting examples.

In some embodiments, excipient particles are comprised of multi-layered particle matrices where in an individual excipient particle is comprised of at least one core matrix and one shell matrix. In some embodiments, a multilayered excipient particle having 3 or more layers is comprised of at least a core matrix, a filler matrix, a shell matrix, or a plurality there for of. In some embodiments, one or more of an excipient's matrix may be comprised of a gas, expedient, lipid, sterol, glucose, sugar, solid, semi-solid, viscoelastic, gelatinous, plastic, amorphous, semi-crystalline, crystalline, liquid crystalline, liquid, porous, porous liquid, a polysaccharide, protein, or a combination thereof.

The polysaccharides in the formulation are selected from the group consisting of polyethylene glycol, polyethylene oxide, starch, hyaluronic acid, gelatin, poly(vinyl alcohol-co-ethylene), poly(vinyl butyral-co-vinyl alcohol-co-vinyl acetate), poly(vinyl chloride-co-vinyl acetate-co-vinyl alcohol), poly-N-para-vinylbenzyl-lactonamide, chondrotin sulphate, dextran, cyclodextrin, polyglycolide, glycolide L-lactide copolymers, glycolide/trimethylene carbonate copolymers, poly-lactides, poly-L-lactide, poly-DL-lactide, L-lactide/DL-lactide copolymers, lactide/tetramethyl-glycolide copolymers, poly-caprolactone, poly-valerolacton, poly-hydroxy butyrate, polyvinyl alcohol, polyhydroxy valerate, poly-N-isopropylacrylamide and lactide/trimethylene carbonate copolymers, polyvinyl pyrrolidone, polyethylene imine, chitosan, carboxymethyl chitosan, chitin, pullulan, dextrose, cellulose, carboxymethyl cellulose, alginate, glucomannan, poly-γ-glutamic acid, poly-propylene glycol, poly-acrylic acid, poly(lactic-co-glycolic acid), poly-caprolactone, poly-valerolactone, poly-hydroxy butyrate, polyvinylpyrrolidone, polyethyleneimine, and lactide/trimethylene carbonate copolymers.

The proteins in the formulation are selected from the group consisting of but not limited too human serum albumin, bovine serum albumin, protamine, transferrin, lactoferrin, fibrinogen, gelatin, mucin, soy protein, apoferritin, ferritin, lectin, gluten, whey protein, prolamines, gliadin, hordein, secalin, zein, and avenin.

In some embodiments of the present disclosure, a excipient particles shell or a plurality of excipient particles shells, may internally and or externally deploy pharmaceutically acceptable stabilizing agents such as but not limited to amphiphilic phospholipids, citric acid, glycols, alcohols, chondritin sulfate, sodium carbonate, lectins, Tris base, cationic surfactants, nonionic surfactants, polyvinyl pyrrolidone, aqueous dispersible co-block polymers, poly gamma glutamic acid, EDTA, chitisin, amine polymers, or glycerin's, cellulose hyaluronic acid, alginate, gelatin, or a combination there fore of.

In some embodiments, an excipient particle is comprised of a durable assembly of smaller nanoparticular excipient units. In some embodiments, smaller nanoparticular excipient units and or assemblies therefore of may be stable at temperatures above about 45.5° C., unstable below about 45° C., or a combination therefore of.

In some embodiments, the excipient system is comprised of a plurality of micron and nanometer sized excipients, some of which are configured to respond to environmental stimuli. Stimuli induced response include, but are not limited to, change in one or more excipient particles external or internal size, structure, function, spatiotemporal function, contents, mobility, morphology, electrical potential, turgid pressure, porosity, free surface area, reactivity, composition, or integrity, or a plurality therefore of.

In some embodiments, the formulation comprising the excipient particle is configured to independently release the therapeutic agents from the core and the shell.

In some embodiments of the present disclosure, a portion of a membrane, shell, core matrix, or a plurality there for of; may be comprised of a functional component capable being stimulated by an external electric, magnetic, or electromagnetic stimuli; preferably located peripherally outside a body of mammalian tissue. In some embodiments of the present disclosure, an excipients functional component may be stimulated to directedly alter the excipients position in spacetime. In some embodiments of the present disclosure, an external stimulus is utilized to induce temperature change within a range of about of 0.1 nm 1 mm in and around a functional component or a plurality there for of. In some embodiments of the present disclosure a functional component is used in conjunction with an external stimulus or a plurality of pulsating external stimuli programmable utilized to alter the structure, composition, chemical, biological, chemical, electrical, physiological, pharmacokinetic, or pharmacodynamic integrity of an excipient, expedient, agent, membrane, tissue, or ingredient proximally located near the functional components, or a plurality therefore of.

In some embodiments, a functional component is comprised of a magnetic, superparamagnetic, thermoelectric, 3 dimensional, 2 dimensional, organic, inorganic, coated, complexed, crystalline, semi crystalline, porous, nonporous, amorphous conductive, or cross-linked lattice, or a plurality there for of.

In some embodiments, excipient particles containing a functional component or a plurality therefore of, may be in contact with a thermal, electrical, or chemically conductive reinforcing matrix connected to the functional component and within or around the excipient, wherein the reinforcing matrix proximally connected to a functional component, or a plurality therefore of; form a conduit capable of reacting to an external magnetic, electric, or electromagnetic stimuli.

In some embodiments external stimuli may be utilized to inductively or conductively effect functional component and structures located in the immediate vicinity. In some embodiments, functional components may act as a wave guide for electromagnetic stimuli.

In some embodiments, multilayered core-shell excipient particles structure may be comprised of at least one with an outer layer comprising a pH responsive outer membrane and encompassing an amount of a first expedient or agent, wherein the outer layer further encapsulates an interior comprised of at least a second interior shell containing an amount of a second expedient or agent encapsulated as the particles core. In some embodiments excipients agents are pharmaceutical ingredients, are pharmaceutical acceptable carriers, or modulating agents.

In some embodiments of the disclosure the pH responsive outer layer may be comprised at least in part, by an acid liable membrane capable of dissolving, swelling, or chemically reacting with proximally located acid surfaces. In preferred embodiments, an acid liable membrane is further capable of reacting with a first expedient, agent or combination therefore of to enhance the bioavailability of the agent or expedient. In some embodiments, acid liable layer may increase the bioavailability of an expedient or agent by increasing its absorption rate. In some embodiments, the absorption rate may increase as a function an acid liable membrane reducing the proximal pH to protect expedient or agent from degradation, catalytically aiding the conversion of expedient or agent to a more bioavailable form, forming a pharmacokinetic or pharmacodynamic coating on proximally located mammalian tissues, or any other means known in prior art, or a combination thereof.

In some embodiments, excipient particles are nanoparticles having an average diameter of about or less than 450 nm. In preferred embodiments, excipient nanoparticles are outer layer's first expedients or agents are anteriorly disposed inside mammalian tissues at a rate of about 1.5 times quicker than expedient or agents contained in particles interior core.

In other embodiments, excipient systems contain a plurality of core shell particles comprising of an outer layer containing a fist expedients or agents capable of selectively inhibiting neuronal cup enzymes, where in particles outer layer first expedients or agents is present in a dose of the system, in concentrations insufficient to inhibit first excipients or agents corresponding liver enzymes.

In some embodiments of the present disclosure, excipient particles are comprised of a core matrix, at least one expedient, and an outer encapsulating membrane. In some embodiments a core matrix is comprised of a network of a pharmaceutically acceptable ingredient and a plurality of nanoparticle excipients.

In some embodiments expedients are comprised of a coalition of multiple excipient particles no larger than about 25 microns in diameter; wherein the coalition is comprised of a plurality of excipient nanoparticles having an individual diameter no greater than about 450 nm and wherein respective surfaces of the excipient nanoparticles within the coalition are discriminately confined to a proximal distance of no greater than about 725 nanometers from another nanoparticle surface contained within the coalition.

In some embodiments, a dose of an abuse limiting, or reduced abuse liability excipient systems are comprised of a plurality of stable excipient nanoparticles and metastable excipient particles. In some embodiments, excipient systems are further comprised of at least one expedient encapsulated in a plurality of stable first nanoparticular excipients and at least one modulating agent contained within a stably dispersed complex of metastable excipient particles, wherein the amount of modulating agent present in metastable excipients is sufficient alter the bioavailability of expedient contained in nanoparticle excipients sufficient to reduce deleterious effects of excessive consumption of the expedient.

In some embodiments of the present disclosure, a dose of an abuse limiting or reduced abuse liability excipient system solution comprised in part, of a metastable complex combined with a pharmaceutically acceptable stabilizing agents such as but not limited to amphiphilic phospholipids, citric acid, glycols, alcohols, chondritin sulfate, sodium carbonate, lectins, Tris base, cationic surfactants, non-ionic surfactants, polyvinyl pyrrolidone (PVP), aqueous dispersible co-block polymers, poly gamma glutamic acid, EDTA, chitosin, amine polymers, or glycerin's, cellulose hyaluronic acid, alginate, gelatin, or other acceptable stabilizing agents know in prior art; or a combination there fore of, to form a metastable excipient particle.

As used herein, the term “metastable” refers to an excipient particle comprised of a dynamic hierarchical assembly of smaller nanoparticle excipients having a mean diameter of about 10-500 nm; and wherein the assembled particles are capable of undergoing a significant change in shape, functionality, morphology or a combination thereof, in response to proximal environmental stimuli. The presence of the stimuli can be employed to elicit in a rapid programmable changes of metastable excipient, such as but not limited to, changes in assembled metastable excipients mean particle size; resulting's in reorganization of the assembly into a plurality of more discrete excipient particles as small as about 1 nm, or growth of the particle to about 1.5 microns in mean diameter or larger.

In some embodiments of the present disclosure, metastable complexes can be formulated to comprise of a complexing expedient, including but not limited to, those comprised of casein, cyclodextrins, other complexing agents known in the art, or a plurality therefore of. In some embodiments of the disclosure, metastable excipient particles are comprised of at least one expedient or agent complexed with a complexing agent and a pharmaceutically acceptable stabilizing agent.

In some embodiments of the present disclosure, an expedients or agents are extracted from herbal tissues and combined with a complex agent to form an herbal extract complex excipient. In some embodiments, an herbal extract complex excipient is comprised of a plurality of expedients or agents derived from herbal tissues. In preferred embodiments multiple excipients or agents comprising extracts containing a plurality of phytocompounds derived from one or more herbal species tissues, are complexed with a complexing agent, to form an excipient complex. Selectively extracted phytocompounds can be utilized to configure the functional properties of the complexes and tailored to produce metastable excipient particles. In some embodiments, excipient complexes containing expedients, agents, or a plurality therefore of may further be combined with one or more pharmaceutically acceptable stabilizing agent to produce a metastable excipient particle or combination thereof.

In some embodiments, metastable excipient particles may be held together by weaker attractive forces or crosslinked by chemical bonds that are reversibly or irreversibly affected by proximal stimuli.

In some embodiments of the present disclosure, metastable excipient particles may be configured to elicit desired conformational changes in the metastable excipients particles structures and programmatically employed to directly or indirectly alter the rate of an excipients delivery and or metabolic profiles of agents, ingredients, expedients, or a combination therefore of, contained within disclosed systems.

In some embodiments nanoparticular excipient systems are comprised of a rapidly released active expedient encapsulated in a plurality of stable first nanoparticular excipients, and a delayed release modulating agent contained within a stably dispersed complex of metastable excipient particles. In other embodiments of the present disclosure, nanoparticular excipient systems are comprised of a delayed release expedient encapsulated in a plurality of stable first nanoparticular excipients and a rapid release modulating agent contained within a stably dispersed complex of metastable excipient particles.

In other embodiments of the present disclosure, nanoparticular excipient systems are comprised of a concomitantly released psychotropic expedient encapsulated in a plurality of stable first nanoparticular excipients and a first agent contained within a stably dispersed complex of metastable excipient particles.

In one embodiment, the present disclosure relates to a complex comprising of cyclodextrins or derivatives therefore of and a plurality of Mitragynine, Paynantheine, Speciogynine, 7-Hydroxymitragynin, Speciogynine, Mitraphylline, Isomitraphylline, Speciogynine, Mitraphylline, Isomitraphylline, Speciophylline, Rhynchophylline, Isorhynchophylline, Ajmalicine, Corynantheidine, Corynoxine A, Corynoxine B, Mitrafoline, Isomitrafoline, Oxindale A, Oxindole B, Speciofoline, Isospeciofoline, Ciliaphylline, Mitraciliatine, Mitragynaline, Mitragynalinic acid, Corynantheidalinic acid.

In some embodiments, it is preferred that compositions comprising of expedient complexes of cyclodextrins or derivatives therefore of, wherein the are comprised of Mitragynine, Paynantheine, Speciogynine, 7-Hydroxymitragynin, Speciogynine, Mitraphylline, Isomitraphylline, Speciogynine, Mitraphylline, Isomitraphylline, Speciophylline, Rhynchophylline, Isorhynchophylline, Ajmalicine, Corynantheidine, Corynoxine A, Corynoxine B, Mitrafoline, Isomitrafoline, Oxindale A, Oxindole B, Speciofoline, Isospeciofoline, Ciliaphylline, Mitraciliatine, Mitragynaline, Mitragynalinic acid, Corynantheidalinic acid.

In some embodiments of the present disclosure, a dose of an abuse limiting or reduced abuse liability excipient system is configured for reducing abuse potential of psychotropic expedients by deploying rapid and delayed delivery excipients comprising off at least one expedient or agent complexed with a plurality of natural or derivatized cyclodextrin complexes; where in the amount of expedients or agents present in the dose is sufficient to achieve desirable cognitive effects within about 10 minutes of consuming the dose and a duration lasting as long as about 11 hours after the dosages consumption and wherein the concentration of the first psychotropic expedient does not exceed 10 mg per a mL of dose solution. In preferred embodiments, the concentration of first psychotropic expedient does not exceed 1 mg per mL of the excipient systems solution. In some embodiments of the present disclosure, a dose of an abuse limiting, or reduced abuse liability excipient system is configured to have a dose capable of inducing desired cognitive effects as soon as 10 minutes and lasting as long as 24 hours in in a consumer subsequent to ingestion of a preponderance of a single dose or multiple dosages by consumers.

In some embodiments of the present disclosure, an excipient system solution is comprised of in part of a plurality of rapid delivery excipients, where in rapid delivery excipients are comprised of an lipophilic expedient complexed with a cyclodextrin; and wherein the complexes are metastable nanoparticles having a water-solubility greater than the lipophilic drugs but less soluble than corresponding drug complexes of then natural α-cyclodextrin and or cyclodextrin derivatives, such as drug complexes of 2-hydroxypropyl-β-cyclodextrin, 2-hydroxypropyl-γ-cyclodextrin, randomLy methylated β-cyclodextrin and sulfobutylether β-cyclodextrin.

Furthermore, some embodiments of the present disclosure pertain to a excipient system solution containing in part, a delayed delivery excipients comprised of an lipophilic expedient complexed with a cyclodextrin wherein the expedient complexes are comprised of metastable, microparticles, nanoparticles, or a combination therefore, having a water-solubility greater than the lipophilic excipient alone and or greater than the corresponding lipophilic expedients complexed with natural β-cyclodextrin and or γ-cyclodextrins.

Some embodiments of the present disclosure pertains to a dose of solution of comprised of at least one natural or derivatized cyclodextrin complexed with an active expedient in the amount of least 0.001 μmole; wherein the expedient(s) are comprised of: 2-aminoethanesulfonic acid, 2-Fluorohmefentanil, 3-Methylfentanyl, 4-Phenylfentanyl, 4-carboethoxyohmefentanil, 14-Cinnamoyloxycodeinone, Acetorphine, Acetylfentanyl, Acetylpropionylmorphine, Actinidine, Aporphine, Ajmalicine, Akuammidine, ψ-Akuammigine, Alfentanil, Alfentanyl, Alfentanil, Amphetamine, Arecoline, Asarone, Ayahuasca extracts, Baicalein, B eta-Casomorphine-7, Benzodiazepine, BDPC, Benzoylmethylecgonine, Brifentanil, Buprenorphine, Butyrfentanyl, Cannabidiol, Cannabidiolic Acid, Cannabinol, Cannabigerol, Cannabichromene, Cannabicyclol, Cannabivarin, Cannabidivarin, Cannabichromevarin, Cannabigerovarin, Cannabigerol Monomethyl Ether, Cannabielsoin, Cannabicitran, Carfentanil, Carnitine, Clonitazene, Codeine, Corynantheidine, Corynoxeine, Corynoxine A, Corynoxine B, Chrysin, Delosperma harazianum, a Dehydromethysticin, Desmethoxyyangoni, Desomorphine, Dezocine, Dextroamphetamine, Diamorphine, Diacetylmorphine, Diacetyldihydromorphine, Dibenzoylmorphine, Dihydrocodein, Dihydroetorphine, Dihydrodesoxy morphine, a Dihydromethysticin, a Dihydrokavain, a Dihydroyangonin, Dipropionate, a Diterpene, Enadoline, an Epicatechin, Ethanol, Etonitazene, Etorphine, Fentanyl, Flavokavain A, Flavokavain B, Flavokavain C, Furanylfentanyl, a Guarana constituent, a Ginsenoside′ a Hemiterpene, a Hydroxyavain, Hydrocodone, Hydromorphone, a Hydroxycorynantheidine, a Hydroxydehydrokavain, a Hydroxymitragynine, a Hydroxyyangonin, Hyperforin, an Iboga alkaloid, Isomitraphylline, Isopteropodine, Kavain, a Kavalactone, Lactucin, Lactucopicrin, Lagochilin, Leonurine, Levoamphetamine, Levorphanol, Lofentanil, Lobeline, Lysergic acid, Lysergic acid diethylamide, Mesembrine, Methadone, Methoxetamine, a Methoxyyangonin, Methoxy-12-hydroxydehydrokavain, Methyldesorphine, Methysticin, Mitragynine, Mitraphylline, Mitajavine, a Monoacetylmorphine, a Monoterpene, Morphine, Morphine dinicotinate, Myrcene, Myristicin, Nicomorphine, Nicotine, N-methylphenethylamine, a Norisoprenoid, N-Phenethyl-14-ethoxymetopon, N-Phenethylnordesomorphine, N-Phenethylnormorphine, Ocfentanil, Ohmefentanyl, Oxycodone, Oxymorphol, Oxymorphone, Papaverine, Paynantheine, Pethidine, Phenaridine, Phenazocine, Phenethylamine, Phenomorphan Phenibut, a Polyterpene, Pukateine, Psilocybin, Remifentanil, a Rhodiola Rosea extract, Rhynchophylline, a Sesterterpene, a Sesquiterpene, a Sesquarterpene, Speciociliatine, Speciogynine, Speciophylline, Sufentanil, a Tetraterpene, a Tetrahydroyangonin, a Tetrahydroalstonine, a Tetrahydrocannabinol, a Tetrahydrocannabinolic acid, a Tetrahydrocannabivarin, Thebaine, Trefentanil, a Triterpene, Voacangine, Yangonin; Yohimbine; an isomer of any of the the expedients; an analogue any of the the expedients; a derivative of any of the the expedients; a salt of any of the said, a metabolite of the expedients; or a combination therefore of.

Preferably in one embodiment of the present disclosure, a dose of solution of comprised of a first excipient composed of at least a natural or derivatized cyclodextrin complexed with at least 0.01 μmoles of comprised of an expedient selected from: 2-Fluorohmefentanil, 3-Methylfentanyl, 4-Phenylfentanyl, 4-carb oethoxyohmefentanil, 14-Cinnamoyloxycodeinone, 7-Hydroxymitragynine, 9-Hydroxycorynantheidine Acetorphine, Acetylfentanyl, Acetylpropionylmorphine, Alfentanyl, Alfentanil, beta-phenyl-gamma-aminobutyric acid, Brifentanil, Buprenorphine, Butyrfentanyl, BDPC, C-8813, Carfentanil, Codeine, Clonitazene, Desomorphine, Diamorphine, Diacetylmorphine, Diacetyldihydromorphine, Dibenzoylmorphine, Dihydrocodein, Dihydroetorphine, Dihydrodesoxy morphine, Dipropionate, Desomorphine, Dezocine, Enadoline, Etonitazene, Etorphine, Fentanyl, Furanylfentanyl, Hydrocodone, Hydromorphone, Ocfentanil, Ohmefentanyl, Oxycodone, Oxymorphol, Oxymorphone, Levorphanol, Lofentanil, Nicomorphine, Nicotine, N-Phenethyl-14-ethoxymetopon, N-Phenethylnordesomorphine, N-Phenethylnormorphine, Methoxetamine, Monoacetylmorphine(s), Methyldesorphine; Morphine dinicotinate, Mitragynine, Morphine, Methadone, Papaverine, Pethidine, Phenomorphan, Phenaridine, Phenazocine, Remifentanil, Speciogynine, Speciociliatine, Sufentanil, Trefentanil, Thebaine, an isomer of any of the the expedients; an analogue any of the the expedients; a derivative of any of the the expedients; a salt of any of the said, a metabolite of the expedients; or a combination therefore of; and a second excipient composed of at least a natural or derivatized cyclodextrin complexed with a second expedient in an amount of least 0.0015 μmoles of an expedient comprised of: Ajmalicine, Caffeine, Cannabidiol, Cannabidiolic Acid, Cannabinol, Cannabigerol, Cannabichromene, Cannabicyclol, Cannabivarin, Cannabidivarin, Cannabichromevarin, Cannabigerovarin, Cannabigerol Monomethyl Ether, Cannabielsoin, Cannabicitran, Corynantheidine, Corynoxeine, Corynoxine A, Corynoxine B, (−)-Epicatechin, 9-Hydroxycorynantheidine, 7-Hydroxymitragynine, Isomitraphylline, Isopteropodine, Methoxetamine, Mitragynine, Mitraphylline, Paynantheine, Rhynchophylline, Speciociliatine, Speciogynine, Speciophylline, a Terpene, a Tetrahydroyangonin, Tetrahydroalstonine, Tetrahydrocannabinol, Tetrahydrocannabinolic acid, Tetrahydrocannabivarin, Tetrahydroalstonine, an isomer of any of the the expedients; an analogue any of the the expedients; a derivative of any of the the expedients; a salt of any of the said, a metabolite of the expedients; or a combination therefore of; and where in the second expedient is different from the fist.

In some embodiments of the present disclosure, an expedients or agents are extracted from herbal tissues and combined with a complex agent to form an herbal extract complex excipient. In some embodiments, an herbal extract complex excipient is comprised of a plurality of expedients or agents derived from one or more herbal species tissues, to produce excipient complexes.

In some embodiments, a pharmaceutically acceptable stabilizing agent is combined with expedient or agents contained in and around an excipient complexes or excipient nanoparticles to form an inhalable excipient delivery system. In some embodiments the inhalable excipient delivery system is comprised of dry powders. In some embodiments the inhalable excipient delivery system is comprised of a readily vaporizable liquid, solution, resin, or syrup, or a plurality there for of.

In some embodiments of the present disclosure, excipient complexes and or nanoparticle excipients are comprised of one or more thermally liable herbally derived expedients, agents, or a plurality there for of. In some embodiments, excipients carrying thermally liable expedients, agents or a combination thereof, are comprised of at least one active expedient or agent that is subject to significant degradation, destabilization, oxidation, transformation, or a plurality therefore of at temperatures of about 70° C. 150° C.

In preferred embodiments of the present disclosure, excipients carrying thermally one or more thermally liable expedients or agents are combined the a pharmaceutically acceptable stabilizing agent to produce a formulation with enhanced thermal stability, where in the formulation is capable of preserving a therapeutically effective portion of the thermally liable expedients or agent's natural chemical functionality in biological tissues, when inhaled at temperatures up to about 500° F.

In some embodiments, inhalable excipient systems are configured to reduce abuse by delivering functionally preserved expedients or agents to pulmonary tissues allowing for rapid onset of desired cognitive effects with increased bioavailability and reducing the concentrations of expedients normally require for oral or other parental methods of administration. In preferred embodiments, expedients or agents are delivered in an inhalable excipient system where in the concentration of an abuse liable excipient is less than 10 mg per mL of excipient system.

In preferred embodiments, an inhalable excipient system includes of a first expedient and an modulating agent comprising an excipient or agent having a relevant IC50 value of about or less than 46 μM effecting CYP3A4, CYP2D, or CYP1A1 enzymes, or a combination there for of; and wherein the modulating agent is present in one dose in amount sufficient to enhance the natural bioavailability of the first expedient.

In one embodiment of the proposed disclosure, full spectrum aqueous dispersible complexes of containing a plurality of herbal derived extracts comprising of at least one psychoactive expedient are preferred. As used herein, the term “full spectrum” refers to an extract rich composition comprising of a plurality of herbal derived compounds that have been separated from their native herbal tissue structures. As used herein, the term “herbal tissue” refers to plant cellular tissues or matrices comprising of but not limited to leaves, stems, seeds, skin, bark, flowers, and roots, or a plurality therefore of.

In a non-limiting example, full spectrum complexes may prepared by first adding complexing agents such as cyclodextrin to a vessel, adding herbal tissues to a container having a porous membrane structure sufficient to prevent leakage of herbal tissues during extraction; adding porous container containing herbal tissues to vessel containing complexing agent; introducing a dissolving fluid to vessel containing herbal tissues and complexation agent in state and quantity insufficient to fully dissolve or disperse the entirety of complexing agent contained within the vessel; heating and agitating the fluid to dissolve or disperse a preponderance of complexing agent while contacting a preponderance of the interior of containers porous membrane while inside the vessel, dissolving a portion of the psychoactive and non-psychoactive excipients residing in the herbal tissues from the interior of the porous container inside the vessel; contacting the dissolved expedients in the fluid with complexing agent to produce an extract complex inside the vessel; cooling the fluid inside the vessel to precipitate a preponderance of complexed herbal extract to the exterior of the porous membrane container; removing the porous membrane container encompassing the herbal tissues from the vessel to separate precipitated complexes; and removing a portion of the precipitate extract complexes from the vessel.

Furthermore, in some embodiments of the disclosure, a mixture of various herbs may be utilized to form aqueous dispersible extract complexes having multiple psychotropic and non-expedients. Optionally after retravel of full spectrum extracts complexes from separation vessel; portions of individual compounds may be further removed based off solvent-selective decomplexation to produce more isolated fractions of desired extracts.

A non-limiting example of such excipients are comprised of at least one psychoactive expedient cannabinoid complexed plurality of cyclodextrins consisting natural and derivatized cyclodextrins consisting of an α-cyclodextrin, β-cyclodextrin, γ-cyclodextrin, or plurality therefore of.

In other embodiments complexing agents are utilized as a means of both controlling delivery rate of an expedient of agent, or a plurality there for of; as well as a means of reducing the negative effects of undesired gastrointestinal irritation caused by the presence of excessive or irritating surfactant. In some embodiments, excipients complexes delivering expedients or agents in and around mammalian gastrointestinal tissues may be combined with pharmaceutically acceptable ingredients to form an ancillary complex with nanoparticle excipients anteriorly delivered, concomitantly administered, or subsequently administered after consumption of complexes and pharmaceutically acceptable ingredients. In some embodiments, excipient complexes co-administered with pharmaceutically acceptable ingredients are configured to react with proximal environmental stimuli inside of mammalian digestive systems.

Reduced Cost Production Systems

Surprisingly it has been found that stable excipient systems disclosed here in can be produced and safely stored at cryogen temperatures as low as about 90° C. for prolonged periods of time. Thus, some embodiments of the present disclosure relate to a nanoparticle excipient system comprising of cryogenically thermostable nanoparticle excipient system. the cryogenically stable excipient systems can have surfaces, shells, cores payloads, or moiety's, or a combination there for of being comprised of stable nanoparticle excipient having reinforcing particle matrices structure comprised of lipids, phospholipids, biodegradable non-lipid polymers, carbohydrate polymers, proteins, organic complexes, inorganic materials, or a combination there for of. Additionally, the cryogenically stable excipient systems can have surfaces, shells, cores payloads, or moiety's, or a combination there for of being comprised of liquid, gelatinous, solid, semisolid, microporous, mesoporous, microporous, amorphous, crystalline, or semi-crystalline matrices, or a combination there fore of.

Some embodiments of the present disclosure pertain cryogenically stable nanoparticular drug delivery system having a plurality of combined nanometric and micronized excipient delivery comprising of at least one active expedient encapsulated in a plurality of stable first nanoparticular excipients and at least one active agent contained within a plurality of dispersed excipient particles stable and or metastable at temperatures up about 45° C. or more. Excipient particles carrying expedients, agents, active ingredients, functional moieties, or a combination therefore of me by be combined form a system comprised of at least one readily delivered excipient and one delayed delivery excipient where in a delayed excipient is capable of actively delivering the excipients payloads comprised of active agents, expedients, ingredients, or a plurality there fore of; in and around mammalian tissues at an average rate of about no less than 240 seconds or more subsequent to the initial delivery of the readily deployable excipients active payload when concomitantly administered.

Variable disproportionation's in rates of delivery of excipients in disclosed systems may be a result of differences in excipient particles sizes, compositions, morphology, functionality arising from responsiveness to thermal, chemical, osmotic, physiological stimuli; or a plurality therefore of.

Surprisingly it has been found that concentrations of cryogenically stable excipient particle systems can be successfully packaged in an inert, readily removable cryogenic matrices while still containing more than about 0.4 wt % residual moisture and or solvent content without resulting in significant loss of function from excipients contained within their initial solutions prior to drying. While the presence of additional protective cryogenic matrices can impart additional coast associated from increased weight of transported product, it has been unexpectedly found that the result of disclosed method and systems allows for the direct packaging of undried excipients that can mitigated the need for expensive and time consuming drying processes, specialty sterile packaging processes, reconstitution processes, or a plurality there fore of; thus allowing for an overall reduced cost of production over known products.

Thus, some embodiments of the present disclosure pertain to a cryogenically stable system comprising of at least a plurality of cryogenically stable nanoparticular excipients contained within a readily removable, protective cryogenic matrix.

Preferably in some embodiments, undried excipient particle systems configured to be stable at cryogenic temperatures can be placed directly in a vessel with carbon dioxide resulting in a purtily excipient particles encompassed by a protective dry ice layer.

It has been found that the combination of a dry ice protective layer produced from substantially sterile source of carbon dioxide can serve as an effective means of preventing undesired degradation of undried excipient particle systems during transportation resulting from exposure to oxidation and or microbial growth during transportation

In some embodiments, undried particles are placed directly in a containing vessel with substantial sterile dry ice. The excipient particle systems may optionally be stirred with dry ice matrices to produce a dispersion of excipient particle systems within the dry ice matrices. Furthermore, a containment vessel containing frozen carbon dioxide matrices and excipient particle systems may be pressurized for a period of time, with optional agitation, sufficient for inducing a reconfiguration of the carbon dioxide crystal structure of matrices in and around the excipient particle systems within the containing vessel; into a substantially more continuous protective dry ice network.

In other embodiments, a vessel containing undried excipient particle systems can be contacted with fluidic carbon dioxide, under optional agitation, and subsequently frozen to produce a plurality of excipient particle systems having a protective dry ice matrix within and around the excipient particles.

In some embodiments of the present disclosure mixtures of undried excipient particle systems and cryogenic protective matrices can be configured to produce readily dispersible aggregate cores of undried excipient particle systems encompassed by a dry ice layer having a preferable thickness of at least 1 cm or more encasing aggregation of undried excipient particle systems, by placing the undried excipient particle systems in a containment vessel having a portion of its interior filled by solid carbon dioxide, and subsequently introducing additional dioxide in and around of undried excipient particle systems within the containment vessel.

In come embodiments of the disclosure undried excipient particle systems incased in a protective dry ice layer is formed into loosely packed fine powers where in the excipient particle and encompassing protective dry ice matrices for a network of incased particles having a mean diameter preferably equal to or less than 1 mm, which may or may not have a final texture resembling powdered snow.

Embodiments wherein undried excipient particle systems incased in a protective dry ice layer may be produce through any of the aforementioned methods, followed by the addition of an agitation step.

Optionally mixtures undried excipient particle systems incased in a protective dry ice layer may further be shaped placed into a mold and additionally subjected to reduced temperatures, reduced pressures, increased pressures, or a combination there for of; within the mold to produce shaped articles.

Furthermore, some embodiments of pertain to a of undried excipient particle systems incased in a protective dry ice layer where in the protective CO2 layer configured to form a substantially continuous shaped article such as a block, sphere, cylinder, pellet or a plurality there for of using any of the for mentioned methods.

More preferably in one embodiment of the present disclosure, desired ratios of undried excipient particle systems incased in a protective dry ice layer mixture can be divided to form precisely dosage article of undried excipient particle systems incased in a protective dry ice layer.

Most preferably in some embodiments of the present disclosure it has been discovered that opacity of the protective dry ice layer in and around the undried excipient particle systems can be configured by any of the previously disclosed methods to produce a protective article having optical properties sufficient to retard exposure the undried excipient particle systems contained within the frozen dry ice matrices to the deleterious effects of ultraviolet radiation during transportation and subsequent processing.

A non-limiting example of precisely dosed articles is a molded pellet are formed from cylindrical bodies of solid carbon dioxide approximately about 0.3 cm in diameter and 4 centimeters in length containing about 150 mg of undried excipient particle systems within a semi-continuous network solid dry ice article.

Surprisingly in some aspects of the present disclosure it has been discovered that production and exportation of federally regulated products such as CBD based products produced from hemp is more advantageous when excipients containing controlled substances are suspended in a protective dry matrix material capable of readily being removed as opposed to known liquid suspension agents. The synergistic benefits of low-cost matrix materials, ubiquity, protective properties, and low energy requirements for separation of excipient particle systems and reclamation processes of protective matrix materials is presently deployed to realize a more versatile and economically advantageous production operation over known methods of processing.

In some embodiments articles of undried excipient particle systems incased in a protective dry ice layer mixture may be directly added to a solution of a predetermined amount with in a container to produce a carbonated beverage.

More advantageously, it has been found that the frozen dry ice protective layer can readily be removed prior to the addition of undried excipient particle systems to a dilution body such as water without the need for energy intensive processes.

A non-limiting example of reduced cost production relates to the production of full spectrum cannabis complexes. Full spectrum cannabis complexes may prepared by first adding complexing agents such as cyclodextrin to a vessel, next adding cannabis herbal tissues to a container having a porous membrane structure sufficient to prevent leakage of herbal tissues during extraction; adding porous container containing herbal tissues to vessel containing complexing agent; introducing a CO2 fluid to vessel containing cannabis herbal tissues and complexation agent in state and quantity insufficient to fully dissolve or disperse the entirety of complexing agent contained within the vessel; heating and agitating the CO2 fluid to dissolve or disperse a preponderance of complexing agent while contacting a preponderance of the interior of containers porous membrane while inside the vessel; dissolving a portion of the psychoactive and non-psychoactive excipients residing in the herbal tissues from the interior of the porous container inside the vessel; contacting the dissolved expedients in the CO2 fluid with complexing agent to produce an extract complex inside the vessel; cooling the fluid inside the vessel to precipitate a preponderance of complexed herbal extracts to the exterior of the porous membrane container; further cooling the vessel to solidify the CO2 fluid; depressurizing the vessel; removing the porous membrane container encompassing the herbal tissues and solid CO2 from the vessel to separate precipitated complexes encased by dry ice; and removing a portion of the precipitate extract complexes and surrounding dry ice matrix from the vessel.

In another non-limiting example, dry ice is in and around cannabis complexes retrieved from the vessel is sublimed to produce a dry cannabis extract complex. The dry extract complex is then dispersed in an aqueous solution containing pharmaceutically acceptable stabilizing agents. The resulting solution mix is then subsequently solidified using liquid nitrogen and added to a mold to containing a ratio of 99 grams dry ice per gram of the solidified aqueous mixture, agitated to disperse solid contents inside the mold, and subsequently cooled to produce a cryogenically stable excipient matrices protected by dry ice. In other embodiments, other protective matrix materials such as but not limited to t-butanol may be optionally employed.

In some preferable embodiments, a composition comprising of said a self-nanoemulsifying drug delivery system (SNEEDS) additionally containing 50 mgs of psychotropic cannabis extracts is administered utilizing a transdermal patch. Transdermal patch allows for continuous and steady rate of release into mammalian circulatory tissues. Steady release transdermal systems combined with said SNEED solutions allows for users to rapidly achieve desired cognitive effects. Likewise, users are able to effectively and abruptly cease effects if undesired cognitive effects are encountered. Additionally, because such transdermal systems can provide steady release over extended periods of time, or other advantages known in the art to transdermal systems, users may avoid consuming unnecessary amounts of psychotropic expedients, and thus reduce the risk of developing tolerance associated with such acts.

### EXAMPLES

While example of the present disclosure allows for the utilization of multiple delivery route formulations without limitation, preferable exemplary embodiments will pertain excipient systems comprising of oral liquid dosages. Disclosed examples are non-limiting in nature.

### Example 1: Reduced Abuse Liability Dose

In a non-limiting example, a dose comprising of a 500 mL aqueous solution containing 60 milligrams of an Mitragyna speciosa extract encapsulated in a plurality of stable quick delivery nanoparticle excipients having an average diameter of about 70 nm; 25 mg of cannabinoid expedients contained within and around a plurality of delayed delivery excipients, comprising of at least 4 mg of cannabidiol carried by a stable dispersed metastable excipient complex. Upon consumption of the dose solution, expedients or modulating agents in quick delivery nanoparticle excipients are absorbed internally by a consumer subsequent to consumption, and the desired cognitive effects onset within 20 minutes of dose administration.

Likewise, delayed delivery excipient systems concomitantly consumed are carried by excipient particle systems having a mean diameter about 30 nm large or more than quick delivery nanoparticle excipients resulting in desired cognitive effects to onset within about 40 to 60 minutes of dose administration. Contents of excipient systems comprising of, but not limited to, structural lipids constructing quick delivery nanoparticular excipients matrices, extracts contained within the quick delivery nanoparticle excipients, or stabilizing pharmaceutical agents; are deployed to simultaneously deliver therapeutic cognitive effects and reduce metabolism effecting delayed delivery excipients payloads. Subsequent absorption of delayed cannabinoids in combination with reduced metabolic clearance is synergistically utilized to increase the bioavailability of absorbed cannabinoids, a resulting an increased distribution into lipophilic storage tissue.

The combined effects result in a portion of cannabinoids becoming prolongedly secreted and metabolically active over a period of about 72 hours to 240 hours or more. Prolongedly administered cannabidiol is utilized to reduce stress related drug seeking behavior related addictive substances effecting opioid receptors, cannabinoid receptors, or other neuroreceptors associated with abusable drugs.

### Example 2: Core Shell Delivery Formulations

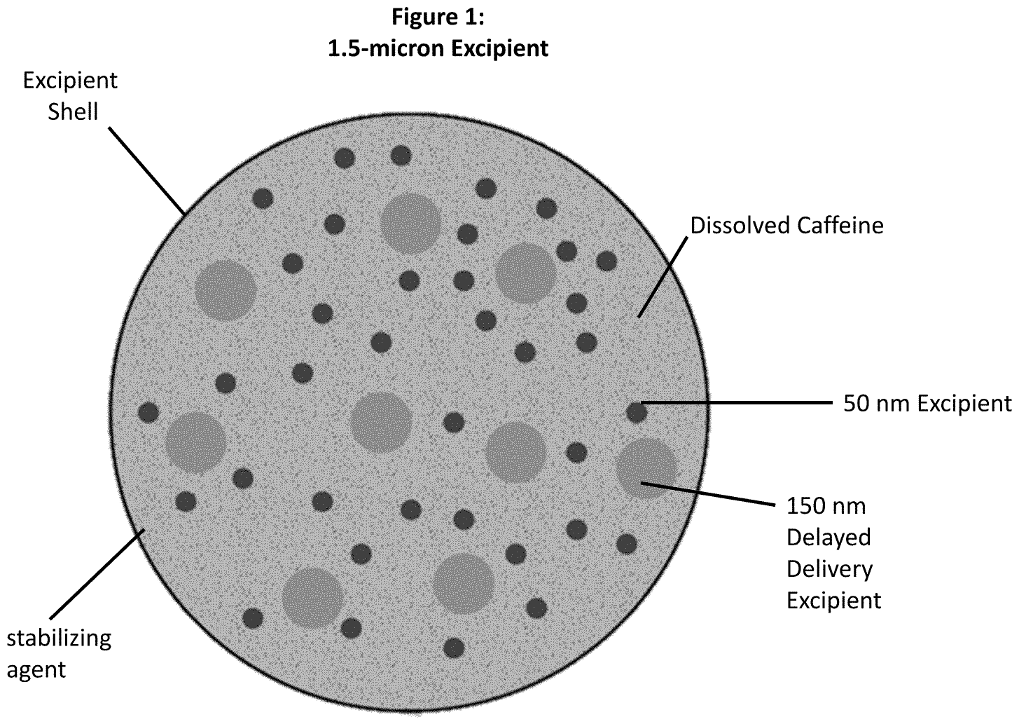

As best shown in FIG. 1, in a non-limiting example, abuse limiting dose solution is comprised of an aqueous 8 oz. beverage containing 150 milligrams of cannabis extract in a stable dispersion semi stable excipient. Excipients are comprised of a semi-stable outer shell comprised of micelles approximately 1.5 microns in diameter, encapsulated a compartmentalized liquid inner phase containing a plurality of nanoparticle excipients. Micron sized excipients interior liquid phases are further comprised of stabilizing solutions collectively containing of approximately 50 milligrams of dissolved caffeine and zwitterionic stabilizers. Encapsulated nanoparticle excipients inside comprise of quick delivery expedient contained in and around nanoparticle excipients having an average diameter of about 50 nm and a delayed delivery nanoparticle excipient having an average diameter of about 150 nanometers. 50 nanometer excipients are comprised of an outer phospholipid lipid membrane and a THC rich cannabinoid payload contained within a low-melting carrier matrix additionally containing multichain tri-glycerides. 50 nanometers are configured to be thermally stable at temperatures of about 45° C. or less but are configured to disassemble rapidly at temperature 50° C. Delayed delivery nanoparticle excipients having an average diameter of about 150 nanometers are comprised of cyclodextrin assemblies and stabilizing agents of propylene glycol and tween 80. Delay delivery nanoparticular complex expedients are predominantly comprised of cannabidiol, with minority complexes containing ethanol, indole alkaloids as well as other complexes containing agents such as surfactants.

Upon consumption of the dose solution, micron sized expedient particles are readily destabilized a consumer's stomach having a pH less than 5, releasing quick and delayed delivery contents. Quick delivery expedients contained within and around 50 nm nanoparticle excipients are absorbed internally by a consumer subsequent to consumption, and the desired cognitive effects onset within 25 minutes of dose administration.

Likewise, a portion of delayed delivery excipient systems concomitantly consumed are carried by excipient particle systems having an initial mean diameter about 100 nm larger than quick delivery nanoparticle excipients, aggregate into larger excipient particles resulting delayed cognitive effects having an onset time of about 60 to 120 minutes of dose administration. Contents of excipient systems comprising of, but not limited to, structural lipids constructing quick delivery nanoparticular excipients matrices, extracts contained within the quick delivery nanoparticle excipients, or stabilizing pharmaceutical agents; are deployed to simultaneously deliver therapeutic cognitive effects and reduce metabolism effecting delayed delivery excipients payloads. Subsequent absorption of delayed cannabinoids in combination with reduced metabolic clearance is synergistically utilized to increase the bioavailability of absorbed cannabinoids, a resulting an increased distribution into lipophilic storage tissue. Delayed delivery Cannabidiol is utilized to further reduce negative psychotropic effects of THC such as anxiety.

These combined effects result in a portion of cannabinoids becoming prolongedly secreted and metabolically active over a period of about 72 hours to 240 hours or more. Prolongedly administered cannabidiol is utilized to reduce stress related drug seeking behavior related addictive substances effecting opioid receptors, cannabinoid receptors, or other neuroreceptors associated with abusable drugs.

In other examples, 8 oz. beverages may be an alcoholic beverage such as beer, wine, or other spirits. The combined effects of alcoholic beverage with a preemptively delivered cannabinoid and delayed delivered cannabinoid or alkaloid combinations, may further utilize quick delivered cannabinoid expedients as a enzymatic, efflux pump, or receptor inhibitor to reduce metabolic clearance of delayed delivered expedients having anxiolytic properties capable of reducing stress induced drug seeking behavior, allowing them to become latently active in mammalian tissue and reduce the effects of alcohol or cannabinoid withdrawal.

### Example 3: Alcoholic Formulations

A non-limiting example of an alcoholic beverage is a wine beverage containing approximately 12% ABV. A 25 mL dispersion of nanoparticle excipients comprising of a plurality of nanoparticle excipients collectively approximately containing 50 mg of two or more herbal extracts comprised of Ajmalicine, Caffeine, Cannabichromene, Cannabidivarin, Cannabicitran, Cannabicyclol, Cannabicyclol, Cannabidiol, Cannabidivarin, Cannabidiolic acid, Cannabigerovarin, Cannabigerol Cannabinol, Cannabivarin, Ciliaphylline, Corynantheidine, Corynantheidalinic acid, Corynoxine A, Corynoxine, 7-Hydroxymitragynin, Isospeciofoline, Isomitrafoline, Isomitraphylline, Isomitraphylline, Isorhynchophylline, Methoxetamine, Mitraciliatine, Mitrafoline, Mitragynine, Mitraphylline Mitragynaline, Mitragynalinic acid, Oxindale A, Oxindole B, Paynantheine, Phenibut, Speciogynine, Speciophylline, Speciogynine, Speciogynine, Rhynchophylline, Speciofoline, Tetrahydrocannabinol, Tetrahydrocannabinolic acid, Tetrahydrocannabivarin, an isomer of any of the the expedients; an analogue any of the the expedients; a derivative of any of the the expedients; a salt of any of the said, a metabolite of the expedients; or a combination therefore of; Is dispersed in 725 mL of wine having an average alcohol content of about 12% ABV.

Alcoholic beverage nanoparticle excipients are further formulated to provide a quick release excipient containing agents and delayed release expedients and agents. Quick release expedients present in wine beverage are comprised of stable lipophilic coated nanoparticle excipients having an average diameter of about 50 nm. Delayed release excipients are further comprised of a plurality of nanoparticles having an average diameter of 200 nm and micron sized particles having an average diameter of about 1500 nm, containing an additional amount of about 50 mgs of expedients, about 25 mgs or more being comprised of Cannabidiol.

A dose of the wine beverage is approximately 125 milliliters. Upon consumption of the dose solution, quick delivery expedients contained within and around 50 nm nanoparticle excipients are absorbed internally by a consumer subsequent to consumption, and the desired cognitive effects onset within 15-35 minutes of dose administration. Expedients or agents contained within quick release nanoparticle excipients are utilized with the wines natural alcohol content to alter the natural effects of alcohol present in the wine. Altered effects may include increased bioavailability of alcohol, increased alertness, reduction in fatigue, or increased metabolic rate to reduce aid in rapid reduction of unwanted excessive intoxication. Additionally, expedient or agent compositions contained within quick release excipient nanoparticles serve to inhibit a portion of delayed delivery excipients payloads, allowing for increased bioavailability, distribution, or latent activity of delayed delivery expedients or agents.

Likewise, a portion of delayed delivery excipient systems concomitantly consumed are carried by plurality of excipient particle systems having an initial mean diameter about 150 nm larger than quick delivery nanoparticle excipients are pharmacokinetically and pharmacodynamically inhibited through competitive or non-competitive, interactions or a combination therefore of; resulting delayed cognitive effects having an onset time of about 60 to 120 minutes of dose administration. Subsequent absorption of delayed cannabinoids in combination with reduced metabolic clearance is synergistically utilized to increase the bioavailability of absorbed cannabinoids, a resulting an increased distribution into lipophilic storage tissue. Delayed delivery Cannabidiol is utilized to further reduce negative psychotropic effects of alcohol such as anxiety, nausea, impulse to continue consuming excessive amounts of alcohol, or other drug seeking behaviors arising from alcohol induced reduced inhibitions.

These combined effects result in a portion of cannabinoids becoming prolongedly secreted and metabolically active over a period of about 72 hours to 240 hours or more. Prolongedly administered cannabidiol is utilized to reduce stress related drug seeking behavior related addictive substances effecting opioid receptors, cannabinoid receptors, or other neuroreceptors associated with abusable drugs.

### Example 4: Thermally Stable Expedient Solutions

A non-limiting example of thermally stable formulations is an 8 oz. caffeinated beverage such as coffee, comprising of a plurality of stable nanoparticle expedients dispersed in a caffeinated beverage at temperatures between 70° C. to 120° C. Nanoparticle excipients have an average diameter ranging between 30 nm 200 nm and may be comprised of lipophilic coated porous nanoparticle, such as silica or polysaccharides, stable complexes of cyclodextrins or casein, or a plurality therefore of Nanoparticle excipients contains expedients in the amount of about 0.001-0.769 μM per 8 fluid oz. of hot caffeine beverage comprising of at least two or more expedients comprised of Mitragynine, Paynantheine, Speciogynine, 7-Hydroxymitragynin, Speciogynine, Mitraphylline, Isomitraphylline, Speciogynine, Mitraphylline, Isomitraphylline, Speciophylline, Rhynchophylline, Isorhynchophylline, Ajmalicine, Corynantheidine, Corynoxine A, Corynoxine B, Mitrafoline, Isomitrafoline, Oxindale A, Oxindole B, Speciofoline, Isospeciofoline, Ciliaphylline, Mitraciliatine, Mitragynaline, Mitragynalinic acid, Corynantheidalinic acid. Ajmalicine, Caffeine, Cannabichromene, Cannabidivarin, Cannabicitran, Cannabicyclol, Cannabicyclol, Cannabidiol, Cannabidivarin, Cannabidiolic acid, Cannabigerovarin, Cannabigerol Cannabinol, Cannabivarin, Ciliaphylline, Corynantheidine, Corynantheidalinic acid, Corynoxine A, Corynoxine, 7-Hydroxymitragynin, Isospeciofoline, Isomitrafoline, Isomitraphylline, Isomitraphylline, Isorhynchophylline, Methoxetamine, Mitraciliatine, Mitrafoline, Mitragynine, Mitraphylline Mitragynaline, Mitragynalinic acid, Oxindale A, Oxindole B, Paynantheine, Phenibut, Speciogynine, Speciophylline, Speciogynine, Speciogynine, Rhynchophylline, Speciofoline, Tetrahydrocannabinol, Tetrahydrocannabinolic acid, Tetrahydrocannabivarin, an isomer of any of the the expedients; an analogue any of the the expedients; a derivative of any of the the expedients; a salt of any of the said, a metabolite of the expedients; or a combination therefore of.

### Example 5: Topical Formulations for Transdermal Application

A non-limiting example of a transdermal formulation is a personal lubricant. A dose of containing approximately 200 mg of psychotropic cannabinoids in a first solution comprised of approximately 25 mL of water and a plurality of nanoparticle excipients containing psychotropic cannabinoid expedients. In some embodiments, nanoparticle excipients are comprised of a mixture of quick release excipients having an average diameter about 40 nm and delayed release expedient encapsulated in a plurality of excipients having an average diameter of 250 nm. The first solution containing nanoparticle excipients is dispersed in a 75 mL of a second solution and agitated sufficiently to form a homogenous lubricating mixture. Preferably on a weight basis, the 100 mL lubricating mixture is comprised of 200 mg of psychotropic cannabinoid expedient contained in nanoparticle excipients totaling about 0.25% or more of the mixture, and is additionally comprised of about 8.0%-27% Glycerin, about 3.0%-6.75% Propylene Glycol, about 5.65%-10.0% Sorbitol, about 0.10%-0.35 5 Potassium Hydroxide, about 0.10%-0.25% Benzoic Acid about 0.2%-1.0% Preservative, about 0.27%-0.8% Hydroxyethylcellulose, about, and about 50% water. Expedients containing psychotropic cannabinoids within 100 mL solution is utilized to formulate a composition for lubricating mucous membranes having an average lubricity range of about 10 to about 470, and a viscosity of about 20-10,000 cp.

In one embodiment a personal lubricating mixture is comprise of about 0.25% psychotropic cannabinoid expedient, about 27% Glycerin, about 6.75% Propylene Glycol, about 10.0% Sorbitol, about 0.10% Potassium Hydroxide, about 0.12% Benzoic Acid about 0.7% Preservative, about 0.6% Hydroxyethylcellulose, about, and about 50% water. Nanoparticle excipients applied, and around erogenous mammalian tissues can allow for onset of desired cognitive effects as soon as 30 minutes and lasting as long as 360 minutes. Formulations utilizing quick release excipients are deployed as a psychotropic lubricating suppository, desire cognitive effects of psychotropic cannabinoids were present within as little as 7 minutes.

In some embodiments of the present disclosure, lubricating psychotropic formulations may also deploy self-nanoemulsifying drug delivery solutions (SNEDDS) compositions contained within a solution of soft gel particle approximately 1-15 microns in average diameter. Subsequent to entering internal mammalian structures, SNEDDS formulations are able to form discriminant nanoparticles of less than 450 nm upon contact of aqueous solutions, such as but not limited to blood.

### Example 6: Quick Delivery Nanoparticle Excipient

40 milliliters of Ethyl Lactate were stirred in 1 milliliters of 200 proof Ethanol with 5 grams of soy Lecithin in a 200 mL flask at 50° C. under vigorous agitation until a first homogenous mixture was formed.

Next 12.7 grams of Sorbitane Monooleate, 12.9 Polysorbate 20, 13.01 grams of Castrol oil and 10.40 grams of Multi Chain Triglyceride oil were stirred at 50° C. homogenously to form a second mixture.

Next 3.11 grams of Mitragyna speciosa extract (50% mitragynine) was stirred in under agitation to form a dispersible Mitragyna speciosa concentrate solution.

The dispersible Mitragyna speciosa concentrate was added drop wise to 2000 mL of water heated to at 60° C. under vigorous agitation at a rate of 5 milliliters per a minute and left to cool to room temperature to form a suspension of stable water soluble fast acting kratom nanoparticles.

Surprisingly it was found that dosages as low as about 2.5 milligram of water soluble Mitragyna speciose nanoparticles were capable of producing noticeable psychotropic effects when consumed by users within 20 minutes of oral or parental administration.

### Example 7: Quick Delivery Nanoparticle Excipient

40 milliliters of Ethyl Lactate were stirred in with 5 grams of soy Lecithin in a 200 mL flask at 50° C. under vigorous agitation until a first homogenous mixture was formed.

Next 12.7 grams of Sorbitane Monooleate, 12.9 Polysorbate 20, 13.01 grams of Castrol oil and 10.40 grams of Multi Chain Triglyceride oil were stirred at 50° C. homogenously to form a second mixture.

Next 2.33 grams of THC cannabis distillate and 0.77 grams of Mitragyna speciosa extract (80% mitragynine) was stirred in under agitation to form a dispersible Mitragyna speciosa concentrate solution.

The dispersible Mitragyna speciosa concentrate was added drop wise to 2000 mL of water heated to at 60° C. under vigorous agitation at a rate of 5 milliliters per a minute and left to cool to room temperature to form a suspension of stable water soluble fast acting psychotropic nanoparticle systems.

Samples ranging from 0.2-50 milliliters of stable water soluble fast acting psychotropic nanoparticle systems were diluted in aqueous solutions for consumption.

Surprisingly it was found that dosages as low as about 0.25 milligram of water soluble Mitragyna speciose nanoparticles were capable of producing noticeable psychotropic effects when consumed by users within 20 minutes of oral or parental administration.

In Some Embodiments, Distillate was a mixture of 99: 1-1:99 tetrahydrocannabinolic acid and Tetrahydrocannabinol

In alternative embodiments, one or more alternative cannabinoids derived from full spectrum hemp extracts used in place of THC.

### Example 8: Quick Delivery Nanoparticle Excipient

40 milliliters of Ethyl Lactate were stirred in with 6 grams of soy Lecithin in a 200 mL flask at 50° C. under vigorous agitation until a first homogenous mixture was formed.

Next 12.7 grams of Sorbitane Monooleate, 12.9 Polysorbate 20, 13.01 grams of Castrol oil and 10.40 grams of Multi Chain Triglyceride oil were stirred at 50° C. homogenously to form a second mixture.

Next 2.89 grams of Mitragyna speciosa extract (80% mitragynine) was stirred in under agitation to form a dispersible Mitragyna speciosa concentrate solution.

In a separate 2000 mL glass vessel, 100 mg of anhydrous caffeine and 4.65 grams of Methyl-β-cyclodextrin were dispersed homogenously dissolved in 1500 mL of water at 70° C. with magnetic stirred hot plate and stir bar.

The dispersible Mitragyna speciosa concentrate was added drop wise to 2000 mL glass vessel heated to at 70° C. under vigorous agitation at a rate of 2.5 milliliters per a minute and left to cool to room temperature to form a suspension of stable water soluble kratom nanoparticles.

Surprisingly it was found that dosages as low as about 1 milligram of water soluble Mitragyna speciosa nanoparticles were capable of producing noticeable psychotropic effects when consumed by users within 20 minutes of oral or parental administration.

In alternative embodiments, the solution was frozen directly after cooling to room temperature and

### Example 9: Delayed Delivery Excipients

3 grams of full spectrum Cannabidiol distillate were diluted in 80 mL of absolute ethanol to form a first mixture.

About 30 grams of 2-Hydroxypropyl-β-cyclodextrin was dissolved in 6000 mL of deionized water at a temperature of 50° C. under light agitation to form a first solution.

The first mixture was added to the first solution under ultrasonic agitation using a 3000 watt ultrasonic homogenizer at rate of 1 mL per a minute for 80 minutes while maintaining a temperature of about 60° C. to form a first delayed delivery CBD suspension. The first delayed delivery suspension CBD was allowed to cool to about room temperature.

Left Blank

### Example 10: Delayed Delivery Nanoparticle Excipient

38 milliliters of Ethyl Lactate were stirred in with 7 grams of soy Lecithin in a 200 mL flask at 50° C. under vigorous agitation until a first homogenous mixture was formed.

Next 12.7 grams of Sorbitane Monooleate, 12.9 Polysorbate 20, 13.01 grams of Castrol oil and 10.40 grams of Multi Chain Triglyceride oil were stirred at 50° C. homogenously to form a second mixture.

Next 3.15 grams of Cannabidiol isolate was stirred in under agitation to form a dispersible Cannabidiol concentrate solution.

The dispersible Cannabidiol concentrate was added drop wise to 900 mL of water heated to at 40° C. under vigorous agitation at a rate of 10 milliliters per a minute and left to cool to room.

Average Particle size was 189 nm.

Left Blank

### Example 11: Delayed Delivery Excipients

3 grams of full spectrum Cannabidiol distillate were diluted in 80 mL of absolute ethanol to form a first mixture.

About 30 grams of 2-Hydroxypropyl-β-cyclodextrin was dissolved in 6000 mL of deionized water at a temperature of 50° C. under light agitation to form a first solution.

The first mixture was added to the first solution under ultrasonic agitation using a 3000 watt ultrasonic homogenizer at rate of 1 mL per a minute for 80 minutes while maintaining a temperature of about 60° C. to form a first delayed delivery CBD suspension. The first delayed delivery suspension CBD was allowed to cool to about room temperature.

### Example 12: Delayed Delivery Nanoparticle Excipient

39 milliliters of Ethyl Lactate were stirred in with 6 grams of soy Lecithin in a 200 mL flask at 50° C. under vigorous agitation until a first homogenous mixture was formed.

Next 12.7 grams of Sorbitane Monooleate, 12.75 Polysorbate 20, 12.9 grams of Castrol oil and 10.6 grams of Multi Chain Triglyceride oil were stirred at 50° C. homogenously to form a second mixture.

Next 3.31 grams of Mitragyna speciosa extract (30% mitragynine) was stirred in under agitation to form a dispersible Mitragyna speciosa concentrate solution.

The dispersible Mitragyna speciosa concentrate was added drop wise to 1200 mL of water heated to at 35° C. under vigorous agitation at a rate of 10 milliliters per a minute and left to cool to room temperature to form a suspension of stable water-soluble delayed delivery psychotropic nanoparticle systems.

Average Particle size was 137 nm.

### Example 13: Delayed Delivery Nanoparticle Excipient

37 milliliters of Ethyl Lactate were stirred in with 9 grams of soy Lecithin in a 200 mL flask at 50° C. under vigorous agitation until a first homogenous mixture was formed.

Next 12.7 grams of Sorbitane Monooleate, 12.75 Polysorbate 20, 12.9 grams of Castrol oil and 10.6 grams of Multi Chain Triglyceride oil were stirred at 50° C. homogenously to form a second mixture.

Next 3.31 grams of Mitragyna speciosa extract (30% mitragynine) was stirred in under agitation to form a dispersible Mitragyna speciosa concentrate solution.

The dispersible Mitragyna speciosa concentrate was added drop wise to 1400 mL of water heated to at 35° C. under vigorous agitation at a rate of 10 milliliters per a minute and left to cool to room temperature to form a suspension of stable water soluble fast acting psychotropic nanoparticle systems.

Average Particle size was 98 nm.

### Example 14: Delayed Delivery Nanoparticle Excipient

39 milliliters of Ethyl Lactate were stirred in with 6 grams of soy Lecithin in a 200 mL flask at 50° C. under vigorous agitation until a first homogenous mixture was formed.

Next 12.9 grams of Sorbitane Monooleate, 12.8 Polysorbate 20, 12.9 grams of Castrol oil and 10.5 grams of Multi Chain Triglyceride oil were stirred at 50° C. homogenously to form a second mixture.

Next 1.5 grams of 85% cannabidiol extract (CBD) and 1.45 grams of L-Tetrahydropalmatine (THP) was stirred in under agitation to form a dispersible CBD/THP concentrate solution.

The dispersible Mitragyna speciosa concentrate was added drop wise to 1000 mL of water heated to at 32° C. under vigorous agitation at a rate of 2 milliliters per a minute and left to cool to room temperature to form a suspension of stable water soluble delayed delivery psychotropic nanoparticle systems.

Average Particle size was 311 nm.

### Example 15: Stable Excipient Systems

3 grams of Mitragyna speciosa extract (45% mitragynine) were diluted in 80 mL of absolute ethanol to form a first mixture.

About 20.7 grams of 2-Hydroxypropyl-β-cyclodextrin was dissolved in 6000 mL of an aqueous solution at a temperature of 45° C. under light agitation to form a first solution.

The first mixture was added to the first solution under ultrasonic agitation using a 3000 watt ultrasonic homogenizer at rate of 2 mL per a minute for 40 minutes while maintaining a temperature at about or below 75° C. to preserve thermo liable compounds from excessive degradation, thus forming a stable Mitragyna speciosa suspension. The stable suspension Mitragyna speciosa was allowed to cool to about room temperature.

### Example 16: Stable Excipient Systems

420 grams of cannabis flower approximately 10% residual moisture were placed in custom polypropylene 100-micron filter bag with 200 grams of 2-Hydroxypropyl-β-cyclodextrin. The filter bag containing cannabis flower and cyclodextrin was placed in a jacketed 5-liter pressure vessel equipped with a removable retaining filter designed to house the filter bag, and a top mounted agitator. About 200 milliliters of ethanol 200 proof was added and the vessel lid was sealed. The vessel was cooled and then injected with liquid carbon dioxide. Subsequent to the vessel becoming filled with liquid CO2, the internal agitator was powered to produce a fluidic agitative force and the vessel was heated to about 48° C. to induce a supercritical CO2 state. The supercritical CO2 state was maintained for about 60 minutes after which the internal agitator was shut off. The vessel was then cooled using a cryogenic fluid and a vent valve at the top of the vessel was opened releasing CO2 gas. The gas was vented while off while the vessel continued to cool until the pressure inside the vessel was reduced to atmospheric pressure. The vessel lid was opened and the removable retaining filter housing the bag filter and herbal tissue were removed. The Cyclodextrin complexed excipients were retrieved from the vessel, producing a substantially dry thermally stable excipient system.

Alternative complexing agents such as Casein complexes can be used as a substitute or in combination with Cyclodextrins. Those skilled in the are will appreciate that alternative solvents selectively deployed for a solvating targeted compounds in corresponding herbal tissues can additionally be employed.

### Example 17: Alcoholic Solutions

In some embodiments, the dispersible Mitragyna speciosa concentrate solution from example 11 were discharged directly into 1500 mL of red wine having an average alcohol content of 12.5% by volume a rate of 2.5 milliliters per a minute at about room temperature to form a stable suspension of delayed delivery kratom nanoparticles having and average particle size of about 250 nm.

### Example 18: Reduced Abuse Liability Dose

50 milliliters of stable water soluble fast acting psychotropic nanoparticle systems from example 7 and 100 milliliters of delayed delivery psychotropic nanoparticle systems from example 14 were added to 600 mL of 11.8 fluid ounces of an aqueous solution comprised of about 6% ethanol by volume, yielding a reduced abuse liability delivery system.

### Example 19: Reduced Abuse Liability Dose

About 38.7 milliliters of quick acting water soluble Mitragyna speciose nanoparticles from example 6 were combined with 100 milliliters of delayed delivery CBD suspension from example 9 were added to about 361 milliliters of an aqueous solution containing about 25 milligrams of caffeine a to produce reduced abuse liability dose.

### Example 20: Reduced Abuse Liability Dose

About 38.7 milliliters of quick acting water soluble Mitragyna speciose nanoparticles from example 6 were combined with 100 milliliters of delayed delivery CBD suspension from example 9 were added to about 361 milliliters of an aqueous solution containing about 25 milligrams of caffeine a to produce reduced abuse liability dose.

### Example 21: Alcoholic Kratom Solutions

In some embodiments, the dispersible Mitragyna speciosa concentrate solution from example 11 were discharged directly into 1500 mL of red wine having an average alcohol content of 12.5% by volume a rate of 2.5 milliliters per a minute at about room temperature to form a stable suspension of delayed delivery kratom nanoparticles having and average particle size of about 250 nm.

### Example 22: Alcoholic Kratom Solutions

In some embodiments, 30 mL Mitragyna speciosa delayed delivery psychotropic nanoparticle systems from example 12 were added to 720 mL of red wine having an average alcohol content of 12.5% under mild agitation to form a delayed delivery systems alcoholic solution.

### Example 23: Low Alcoholic Kratom Solutions

In some embodiments, the dispersible Mitragyna speciosa concentrate solution from example 11 were discharged directly into 1500 mL of red wine having an average alcohol content of about or less than 4.5% by volume a rate of 2.5 milliliters per a minute at about room temperature to form a stable suspension of delayed delivery kratom nanoparticles having and average particle size of about 250 nm.

### Example 24: Low Alcoholic Kratom Solutions

In some embodiments, 30 mL Mitragyna speciosa delayed delivery psychotropic nanoparticle systems from example 12 were added to 720 mL of red wine having an average alcohol content of about or less than 4.5% under mild agitation to form a delayed delivery systems alcoholic solution.

### Example 25: Psychotropic Topical Lubrication Solution

25 mL of stable water soluble fast acting psychotropic nanoparticle systems from example 7 were dispersed in 975 mL of viscoelastic liquid mictured comprised of about 27% Glycerin, about 6.75% Propylene Glycol, about 10.0% Sorbitol, about 0.10% Potassium Hydroxide, about 0.12% Benzoic Acid about 0.7% Preservative, about 0.6% Hydroxyethylcellulose, and about 50% water; under mild agitation to form a psychotropic topical lubrication solution.

### Example 26: Reduced Abuse Liability Psychotropic Topical Lubrication Solution

20 mL of stable water soluble fast acting psychotropic nanoparticle systems from example 7 and 30 mL of delayed delivery systems from example 14 were dispersed in 950 mL of viscoelastic liquid mictured comprised of about 27% Glycerin, about 6.75% Propylene Glycol, about 10.0% Sorbitol, about 0.10% Potassium Hydroxide, about 0.12% Benzoic Acid about 0.7% Preservative, about 0.6% Hydroxyethylcellulose, and about 50% water; under mild agitation to form a reduced abuse liability psychotropic topical lubrication solution.

### Example 27: Reduced Abuse Liability Opioid Dosage

5 mL of stable of water-soluble delayed delivery psychotropic nanoparticle systems from example 14 were frozen and lyophilized to produce a dry delayed delivery psychotropic nanoparticle system. Dried delay delivery psychotropic nanoparticle systems were blended with 4 mg of oxycodone hydrochloride and pressed into a pill to produce a reduced abuse liability dosage.

### Example 28: Reduced Abuse Liability Opioid Solution

5 mL of stable of water-soluble delayed delivery psychotropic nanoparticle systems from example 14 were dispersed into a 15 mL aqueous solution containing 200 micrograms of Fentanyl to produce an Intervenors reduced abuse liability opioid solution.

### Example 29: Edible Quick Delivery Matrices

25 mL of stable of quick delivery psychotropic nanoparticle systems from example 6 were dispersed into a gelatin solution, cast into a mold, and solidified to produce a stable edible matrix impregnated with dispersed quick delivery psychotropic nanoparticle systems.

### Example 30: Chewable Quick Delivery Nanoparticle Excipient

25 mL of stable of quick delivery nanoparticle excipient solutions from example 7 were sprayed onto and around 94 grams of chewing gum having a moisture content of about 6% by weight % or less. The gum and quick delivery mitragynine nanoparticle excipient solution was left to stand for a about 30 minutes to absorb into the porous gum matrices, producing a chewable quick delivery nanoparticle delivery system.

### Example 31: Reduced Abuse Liability Amphetamine Dosage

5 mL of stable of water-soluble delayed delivery psychotropic nanoparticle systems from example 14 were frozen and lyophilized to produce a dry delayed delivery psychotropic nanoparticle system. Dried delayed delivery psychotropic nanoparticle systems were blended with 30 mg of Lisdexamfetamine of pressed into a pill to produce a reduced abuse liability dosage.

### Example 32: Reduced Abuse Liability Opioid Solution

9 mL of stable of water-soluble delayed delivery psychotropic nanoparticle systems from example 7 were frozen and lyophilized to produce a dry delayed delivery psychotropic nanoparticle system. Dried delayed delivery psychotropic nanoparticle systems were blended with 15 mg milligram mixture having active drug ingredients comprising about 25% levoamphetamine salts and 75% dextroamphetamine salts, and subsequently pressed into a pill to produce a reduced abuse liability dosage.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A delivery composition, comprising:
a plurality of first nanoparticle excipients each containing an active compound; and
a plurality of second nanoparticle excipients each containing a modulating agent.

2. The delivery composition of claim 1, further comprising:
water; and
wherein the concentration of the active compound is less than 1 mg/mL.

3. The delivery composition of claim 1, wherein each of the first nanoparticle excipients and each of the second nanoparticle excipients has a diameter of less than 450 nm.

4. The delivery composition of claim 1, further comprising:
microparticles having a diameter of less than 25 urn each containing a plurality of both the first nanoparticle excipients and the second nanoparticle excipients.

5. The delivery composition of claim 1, wherein a mean diameter of the first nanoparticle excipients is smaller than a mean diameter of the second nanoparticle excipients.

6. The delivery composition of claim 5, wherein the active compound includes tetrahydrocannabinol and the modulating agent includes cannabidiol.

7. The delivery composition of claim 5, wherein the active compound includes an opioid and the modulating agent is selected from a group consisting of a μ-opioid agonist, a partial μ-opioid agonist, an opioid antagonist, and a combination thereof.

8. The delivery composition of claim 5, wherein the mean diameter of the plurality of first nanoparticle excipients is between 25 nm and 75 nm, and the mean diameter of the plurality of second nanoparticle excipients is at least 75 nm greater than the mean diameter of the plurality of first nanoparticle excipients.

9. The delivery composition of claim 5, wherein the mean diameter of the plurality of second nanoparticle excipients is at least 150 nm greater than the mean diameter of the plurality of first nanoparticle excipients.

10. The delivery composition of claim 1, wherein the first nanoparticle excipients include THC and the second nanoparticle excipients include Mitragyna speciose extract.

11. The delivery composition of claim 1, wherein the modulating agent is selected from a group consisting of a CYP-2C9 inhibitor having an IC50 value about or less than 32. 1±3.7 μM, a CYP-2D6 inhibitor having an IC50 value about or less than 27.4±5.3 μM, a CYP-3A4 inhibitor having an IC50 value about or less than 43.2±6.2 μM, a CYP450 inhibitor having an IC50 value about or less than 43.2±6.2 μM, a P-glycoprotein inhibitor, and a UG72B7 inhibitor.

12. The delivery composition of claim 1, wherein the modulating agent is an inhibitor or an inducer in an amount sufficient to increase the bioavailability of the active compound.

13. The delivery composition of claim 1;
wherein the plurality of first nanoparticle excipients and the plurality of second nanoparticle excipients form coalitions of multiple excipient particles no larger than about 25 μm in diameter; and
wherein the coalitions are comprised of a plurality of excipient nanoparticles having an individual diameter no greater than about 450 nm and wherein respective surfaces of the nanoparticle excipients within each of the coalitions are discriminately confined to a proximal distance of less than about 725 nanometers from another nanoparticle surface contained within the coalition.

14. The delivery composition of claim 1, wherein the plurality of first nanoparticle excipients are stable and the plurality of second nanoparticle excipients are metastable and contain enough of the modulating agent sufficient to increase the bioavailability of the active compound.

15. The delivery composition of claim 1, wherein the active compounds lipophilic and the plurality of first nanoparticle excipients include cyclodextrin.

16. The delivery composition of claim 1, the plurality of first nanoparticle excipients are subject to at least one of degradation, destabilization, oxidation, and transformation at temperatures between about 70° C. and 150° C.

17. The delivery composition of claim 1, wherein the plurality of first nanoparticle excipients and the plurality of second nanoparticle excipients are configured to be inhaled.

18. A delivery composition, comprising:
a plurality of nanoparticle excipients;
a plurality of microparticle excipients containing cannabidiol;
water; and
ethanol.

19. The delivery composition of claim 13, wherein the concentration of cannabidiol is between 150 and 250 mg/L, and a mean diameter of the plurality of microparticle excipients is between 1 μm and 2 μm.

20. A delivery composition, comprising:
(a) a dispersible concentrate configured for forming upon contact with an aqueous solution particles of a mean diameter of Jess than about 450 nm;
the dispersible concentrate further comprising:
(i) at least one surfactant;
(ii) at least one solid component at room temperature;
and
(iii) an amphiphilic solvent;
(b) at least one Mitragyna speciosa compound selected from the group consisting of Ajmalicine, Akuammigine, Ciliaphylline, Corynantheidine, Corynoxeine, Corynoxine A, Corynoxine B, Epicatechin, 9-Hydroxycorynantheidine, 7-hydroxymitragynine, Isomitraphylline, Isomitrafoline, Isopteropodine, Isorhynchophylline, Isospeciofoline, Mitraciliatine, Mitragynine, Mitragynine oxindole B. Mitrafoline, Mitraphylline, Mitraversine, Paynantheine, Rhynchophylline, Speciociliatine, Speciofoline, Speciogynine, Speciophylline, Stipulating Tetrahydroalstonine, a corresponding analog, metabolite, isomer and a combination thereof, the at least one Mitragyna speciosa compound present in an amount sufficient to increase the bioavailability of the composition.
